(19) United States
(12) Patent Application Publication
(10) Pub. No.: US 2007/0065466 A1
(43) Pub. Date: Mar. 22, 2007

(54) Clostridium difficile vaccine

(75) Inventors: Henry J. Windle, Dublin (IE); Rachael Doyle, Dublin (IE); Dermot Kelleher, Dublin (IE); James Bernard Walsh, Dublin (IE); Deirdre Ni Eidhin, Dublin (IE)

Correspondence Address: JACOBSON HOLMAN PLLC, 400 SEVENTH STREET N.W., SUITE 600, WASHINGTON, DC, 20004, US

(73) Assignees: The Provost, Fellows and Scholars of the College of the Holy and Undivided Trinity of Queen; Elizabeth, near Dublin

(21) Appl. No.: 11/409,261

(22) Filed: Apr. 24, 2006

Related U.S. Application Data
(63) Continuation of application No. 10/068,870, filed on Feb. 11, 2002, now abandoned.

(30) Foreign Application Priority Data
Feb. 9, 2001 (IE) 2001/0137

(51) Int. Cl. A61K 39/08

(52) U.S. Cl. 424/247.1

(57) ABSTRACT

A vaccine for the treatment or prophylaxis of C. difficile associated disease comprises a C. difficile gene or a C. difficile peptide/polypeptide or a derivative or fragment or mutant or variant thereof which is immunogenic in humans. The gene encodes a C. difficile surface layer protein, SlpA or variant or homologue thereof. The peptide/polypeptide is a C. difficile surface layer protein, SlpA or variant or homologue thereof. The vaccine may comprise a chimeric nucleic acid sequence.

This is a Continuation of application Ser. No. 10/068,870, filed Feb. 11, 2002.

### INTRODUCTION

The invention relates to vaccines to provide immunological protection against C. difficile infection.

### BACKGROUND

Clostridium difficile is a common nosocomial pathogen and a major cause of morbidity and mortality among hospitalised patients throughout the world [Kelly et al., 1994]. Outbreaks of C. difficile have necessitated ward and partial hospital closure. With the increasing elderly population and the changing demographics of the population, C. difficile is set to become a major problem in the 21st century. The spectrum of C. difficile diseases range from asymptomatic carriage to mild diarrhoea to fulminant pseudomembranous colitis. Host factors rather than bacterial factors appear to determine the response to C. difficile [Cheng et al., 1997; McFarland et al., 1991; Shim et al., 1998].

Reports indicate that hypogammaglobulinaemia in children appears to predispose to the development of disease due to C. difficile and that therapy with intravenously administered gamma globulin can be associated with the clinical resolution of chronic relapsing colitis due to C. difficile disease [Leung et al., 1991; Pelmutter et al., 1985]. A study by Mulligan et al. [1993] found elevated levels of immunoglobulins reactive with C. difficile in asymptomatic carriers as opposed to symptomatic patients. Recently it has been shown that patients who became colonised with C. difficile who had relatively low levels of serum IgG antibody against toxin A had a much greater risk of developing C. difficile diarrhoea [Kyne et al., 2000].

It is clear that any advance in the understanding of C. difficile disease and methods of preventing or treating C. difficile diarrhoea (CDD) and other related diseases will be of major therapeutic potential.

### STATEMENTS OF INVENTION

According to the invention there is provided a vaccine for the treatment or prophylaxis of C. difficile associated disease, the vaccine comprising a C. difficile gene or a C. difficile peptide/polypeptide or a derivative or fragment or mutant or variant thereof which is immunogenic in humans.

The invention also provides a vaccine for the treatment or prophylaxis of C. difficile associated disease, the vaccine comprising a C. difficile gene or C. difficile peptide/polypeptide or a derivative or fragment or mutant or variant thereof to which immunoreactivity is detected in individuals who have recovered from C. difficile infection.

Preferably the gene encodes a C. difficile surface layer protein, SlpA or variant or homologue thereof.

Preferably the peptide/polypeptide is a C. difficile surface layer protein, SlpA or variant or homologue thereof.

Most preferably the vaccine comprises a chimeric nucleic acid sequence. Preferably the chimeric nucleic acid sequence is derived from the 5′ end of the gene, encoding the mature N-terminal moiety of SlpA from C. difficile.

In one embodiment of the invention the vaccine comprises a chimeric peptide/polypeptide. Preferably the amino acid sequence of the chimeric peptide/polypeptide is derived from the mature N-terminal moiety of SlpA from C. difficile.

Preferably the vaccine of the invention contains an amino acid sequence SEQ ID No. 1 or a derivative or fragment or mutant or variant thereof.

Preferably the vaccine contains an amino acid sequence SEQ ID No. 2 or a derivative or fragment or mutant or variant thereof.

In one embodiment of the invention the vaccine contains a nucleotide sequence SEQ ID No. 3 or a derivative or fragment or mutant or variant thereof; a nucleotide sequence SEQ ID No. 4 or a derivative or fragment or mutant or variant thereof; a nucleotide sequence SEQ ID No. 5 or a derivative or fragment or mutant or variant thereof; a nucleotide sequence SEQ ID No. 6 or a derivative or fragment or mutant or variant thereof; a nucleotide sequence SEQ ID No. 7 or a derivative or fragment or mutant or variant thereof; a nucleotide sequence SEQ ID No. 8 or a derivative or fragment or mutant or variant thereof; a nucleotide sequence SEQ ID No. 9 or a derivative or fragment or mutant or variant thereof or a nucleotide sequence SEQ ID No. 10 or a derivative or fragment or mutant or variant thereof.

Preferably the vaccine of the invention is in combination with at least one other C. difficile sub-unit.

The invention provides a vaccine for the treatment or prophylaxis of C. difficile associated disease, the vaccine comprising the mature N-terminal moiety of a surface layer protein, SlpA of C. difficile or variant or homologue thereof which is immunogenic in humans.

Most preferably the N-terminal moiety of SlpA contains an amino acid sequence SEQ ID No. 1.

In one embodiment of the invention the N-terminal moiety of SlpA contains an amino acid sequence SEQ ID No. 2.

The invention also provides a vaccine for the treatment or prophylaxis of C. difficile associated disease, the vaccine comprising an immunodominant epitope derived from a C. difficile gene or a C. difficile peptide/polypeptide or a derivative or fragment or mutant or variant thereof which is immunogenic in humans.

Preferably the vaccine of the invention comprises a pharmaceutically acceptable carrier. Most preferably the vaccine is in combination with a pharmacologically suitable adjuvant. Ideally the adjuvant is interleukin 12. Alternatively the adjuvant may be a heat shock protein.

In one embodiment of the invention the vaccine comprises at least one other pharmaceutical product.

The pharmaceutical product may be an antibiotic, selected from one or more metronidazole, amoxycillin, tetracycline or erythromycin, clarithromycin or tinidazole.

In one embodiment of the invention the pharmaceutical product comprises an acid-suppressing agent such as omeprazole or bismuth salts.

The vaccine of the invention may be in a form for oral administration, intranasal administration, intravenous administration or intramuscular administration.

In one embodiment of the invention the vaccine includes a peptide delivery system.

The invention also provides an immunodominant epitope derived from a C. difficile gene or a C. difficile peptide/polypeptide or a derivative or fragment or mutant or variant thereof. Preferably the C. difficile peptide/polypeptide contains an amino acid sequence SEQ ID No. 1 or SEQ ID No. 2 or a derivative or fragment or mutant or variant thereof.

In one embodiment of the invention the C. difficile peptide/polypeptide contains an amino acid sequence SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10 or a derivative or fragment or mutant or variant thereof.

The invention further provides a chimeric nucleic acid sequence derived from the 5′ end of the slpA gene encoding the mature N-terminal moiety of SlpA from C. difficile which is immunogenic in humans.

The invention also provides a chimeric peptide/polypeptide wherein the amino acid sequence of the chimeric peptide/polypeptide is derived from the mature N-terminal moiety of SlpA from C. difficile.

The invention provides a C. difficile peptide comprising SEQ ID No. 1 or SEQ ID No. 2 or SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10.

One aspect of the invention provides for the use of a C. difficile gene or a C. difficile peptide/polypeptide or a derivative or fragment or mutant or variant thereof which is immunogenic in humans in the preparation of a medicament for use in a method for the treatment or prophylaxis of C. difficile infection or C. difficile associated disease in a host.

Preferably the medicament which is prepared is a vaccine of the invention.

The invention also provides a method for preparing a vaccine for prophylaxis or treatment of C. difficile associated disease, the method comprising;
obtaining a C. difficile gene or a C. difficile peptide/polypeptide or a derivative or fragment or mutant or variant thereof which is immunogenic in humans; and
forming a vaccine preparation comprised of said gene or peptide/polypeptide or derivative or fragment or mutant or variant, which is suitable for administration to a host and which when administered raises an immune response.

Preferably the C. difficile peptide/polypeptide contains an amino acid sequence SEQ ID No. 1 or SEQ ID No. 2 or a derivative or fragment or mutant or variant thereof.

Most preferably the C. difficile gene contains an amino acid sequence SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10 or a derivative or fragment or mutant or variant thereof.

The invention further provides a method for prophylaxis or treatment of C. difficile associated disease, the method comprising;
obtaining a C. difficile gene or a C. difficile peptide/polypeptide or a derivative or fragment or mutant or variant thereof which is immunogenic in humans;
forming a vaccine preparation comprised of said gene or peptide/polypeptide or derivative or fragment or mutant or variant, and
administering the vaccine preparation to a host to raise an immune response.

One aspect of the invention provides monoclonal or polyclonal antibodies or fragments thereof, to a C. difficile peptide/polypeptide or a derivative or fragment or mutant or variant thereof which is immunogenic in humans.

Another aspect of the invention provides monoclonal or polyclonal antibodies or fragments thereof, to C. difficile peptide/polypeptide or a derivative or fragment or mutant or variant thereof to which immunoreactivity is detected in individuals who have recovered from C. difficile infection.

The invention also provides purified antibodies or serum obtained by immunisation of an animal with a vaccine of the invention.

The invention provides the use of the antibodies or fragments of the invention in the preparation of a medicament for treatment or prophylaxis of C. difficile infection or C. difficile associated disease.

Preferably the antibodies or serum are used in the preparation of a medicament for treatment or prophylaxis of C. difficile infection or C. difficile associated disease.

Most preferably the antibodies or fragments or serum of the invention are used in passive immunotherapy for established C. difficile infection.

In one embodiment of the invention the antibodies or fragment or serum of the invention are used for the eradication of C. difficile associated disease.

The invention also provides use of interleukin 12 as an adjuvant in C. difficile vaccine.

The invention further provides use of humanised antibodies or serum for passive vaccination of an individual with C. difficile infection.

### BRIEF DESCRIPTION OF THE DRAWINGS

The invention will be more clearly understood from the following description thereof given by way of example only with reference to the accompanying figures, in which:—

FIG. 1A is a Western blot showing recognition of antigens from a crude extract of C. difficile 171500 (PCR type 1) by serum antibodies from a patient infected with this strain. Lane 1: Pre-infection; Lane 2: Early acute; Lane 3: Late acute; Lane 4: Convalescent;

FIG. 1B is a Western blot showing recognition of antigens from a crude extract of C. difficile 170324 (PCR type 12) by serum antibodies from a patient infected with this strain. Lane 1: Pre-infection; Lanes 2-5: Acute; Lanes 6-7: Convalescent;

FIG. 2. is a Western blot showing recognition of antigens from two C. difficile strains of different type by serum from convalescent patients.
Lane 1: Strain 170324 (PCR type 12), crude antigen preparation
Lane 2: Strain 170324, surface layer protein preparation
Lane 3: Strain 171500 (PCR type 1), crude antigen preparation
Lane 4: Strain 171500, surface layer protein preparation.
Molecular mass markers (kDa) are shown on the left; and

FIG. 3 is an SDS-PAGE gel showing crude SLP preparations from selected strains of C. difficile. The gel contains 12% acrylamide, and has been stained for protein with Coomassie Blue. Each lane contains 5 μg of protein. Molecular weight markers are shown on the left.
Lane 1: 171500 (PCR type 1)
Lane 2: 172450 (PCR type 5)
Lane 3: 170324 (PCR type 12)
Lane 4: 171448 (PCR type 12)
Lane 5: 171862 (PCR type 17)
Lane 6: 173644 (PCR type 31)
Lane 7: 170444 (PCR type 46)
Lane 8: 170426 (PCR type 92)

### DETAILED DESCRIPTION OF THE INVENTION

Two antigenic peptides containing SEQ ID No. 1 and SEQ ID No. 2, associated with two common infecting types of C. difficile, were found to be immunogenic in humans. The antigenic peptides were found to induce a strong immune response in individuals who recover from C. difficile infection. Individuals who have recovered from C. difficile infection are those individuals who have been exposed to C. difficile or something strongly related and have recovered. This includes individuals where a carrier state exists in that the C. difficile infection has not and will not necessarily become clinically significant.

These antigenic peptides were found to be products of the slpA gene from C. difficile which is the structural gene for the surface layer protein, SlpA. The gene or its products are therefore ideal candidates for the preparation of vaccines against C. difficile.

Surface layer proteins (SLPs), also known as S-layers or crystalline surface layers, are associated with a wide range of bacterial species. They form a 2-dimensional array, which covers the surface of the cell completely, and grows with the cell [Sleytr et al., 1993]. The molecular weight can range from 40 000 to 200 000 Da. The proteins are typically acidic, contain a large proportion of hydrophobic amino acid residues, and have few or no sulphur-containing amino acid residues. Glycosylated S-layer proteins occur in some species. The precise function of S-layers is not always known, but since they comprise approximately 15% of the cell protein, it seems likely that they are important for in vivo functioning of the organism. In Gram positive organisms, the SLP has been shown to delay or prevent the excretion of degradative enzymes from the cell to the outside milieu, and may thereby create a space analagous to the periplasmic space of Gram negative bacteria. Many pathogenic species possess SLPs, which have been ascribed functions such as antiphagocytosis (Campylobacter fetus), and inhibition of complement-mediated killing (Aeromonas salmonicida).

Kawata et, al. [1984] described the SLPs of Clostridium difficile. They showed the S-layer to be composed of 2 polypeptides, and demonstrated size heterogeneity for the polypeptides from different strains. Delmée et al. [1986] showed that crude extracts from C. difficile strains of different serotype showed different polypeptide profiles in SDS-PAGE. Poxton et al. [1999] made similar observations using purified SLP preparations. Slide agglutination [Delmée et al., 1990] has identified 21 different serotypes, apparently distinguished by the heterogeneity of the SLP.

Pantosti et al. [1989] isolated C. difficile from a number of patients with antibiotic-associated diarrhoea, and prepared SLPs from them. Cerquetti et al. [2000] published N-terminal sequences of SLPs from several strains, indicating wide differences between strains. In 2000 the complete DNA sequence of the C. difficile genome was published (available at web address http://www.sanger.ac.uk/Projects/C_difficile/).

The peptides of the invention were found to be encoded by a single open reading frame (ORF) named slpA from C. difficile. The peptides identified in our clinical study correspond to a lower molecular weight moiety of the slpA gene product. Since an immune response is also mounted against a higher molecular weight slpA gene product (FIG. 2), this entity may also be included in a vaccine.

The slpA gene has been sequenced from a number of strains corresponding to different PCR types. The sequences of strains 171500 (PCR type 1)(NCIMB 41081; PHLS R13537), 172450 (PCR type 5)(PHLS R12884), 170324 (PCR type 12) (NCIMB 41080; PHLS R12882), 171448 (PCR type 12) (PHLS R13550), 171862 (PCR type 17) (PHLS R13702), 173644 (PCR type 31) (PHLS R13711), 170444 (PCR type 46) (PHLS R12883) and 170426 (PCR type 92) (PHLS R12871) with translations thereof are given in Appendices 1 to 8. Substantial variation in nucleotide and predicted amino acid sequence was found between strains of PCR types 1, 5, 12, 17 and 31. The genes from strains of PCR types 46 and 92 are almost identical in sequence to those of PCR type 12. When the DNA sequences of genes of different strains within a PCR type are compared, the sequences are almost if not quite identical, indicating that the potential for variation is not infinite. These findings are in agreement with serotyping studies [Delmée et al., 1986, 1990], and indicate that the production of an effective vaccine based on the slpA product is feasible. In this respect, the present invention includes all variant slpA genes and their products, individually and combined, fragments of them, and their mutants and derivatives.

One aspect of the invention provides the combination of immunodominant eptopes from the slpA gene products from various serotypes into a single vaccine. In this way a single vaccine may be used to immunise against several different C. difficile strains.

The most common PCR types isolated from infections in the clinical study carried out at St. James's Hospital, Dublin, Ireland were PCR types 1 and 12. However, a vaccine which elicits an intense antibody response against many infecting types would be therapeutically very valuable. Recombinant DNA chimera, or several chimeras, encoding contiguous immunodominant epitopes may be made for use in the vaccine. The recombinant DNA may serve as the active component in a vaccine, or may be inserted into an appropriate expression system for the generation of a chimeric peptide vaccine in a suitable host.

Chimeras can be generated by PCR amplification of the DNA encoding peptide regions of interest, incorporating cleavage sites for restriction endonucleases into the primers. The amplified fragments can thus be cleaved to generate compatible ends, and spliced together to create chimeras.

The dominant epitopes may be identified by cleavage of the slpA products into fragments by agents which cleave at known sites, and by immunoblotting with homologous patient serum. Immunodominant peptides may be tested for their capacity to stimulate T-cell proliferative responses in vitro, using mouse splenic T-cells.

DNA vaccination involves immunisation with recombinant DNA encoding the antigen or epitope of interest, cloned in a vector which promotes high level expression in mammalian cells. Typically, the vector is a plasmid vector which which also replicates in a procaryotic vector such as Escherichia coli, so that the DNA can be produced in quantity. Following immunisation, the plasmid enters a host cell, where it remains in the nucleus, and directs synthesis of the recombinant polypeptide. The polypeptide stimulates the production of neutralising antibodies, as well as activating cytotoxic T-cells.

Using a DNA vaccine, it may be necessary to modify the DNA sequence to take account of codon usage in humans. The G+C content of mammalian DNA is much higher than that of C. difficile. The generation of such synthetic DNA molecules, essentially containing numerous silent mutations, is within the scope of the invention.

A peptide vaccine will ideally be made using recombinant peptides. Similar considerations apply as in the generation of a DNA vaccine with regard to expression in a different host, such as Escherichia coli, which has a different codon usage pattern to C. difficile. Problems of expression may be overcome by the use of a special host strain which carries additional copies of rare tRNAs (e.g. E. coli BL21-CodonPlus™-RIL from Stratagene), or by using de novo synthesis of a DNA segment carrying silent mutations which will enable normal expression in E. coli. There are many expression systems which are likely to allow high-level expression of slpA genes in E. coli. An example is the pBAD/Thio TOPO vector of Invitrogen, in which expressed genes are under control of the arabinose promoter, which is subject to positive and negative control, enabling very tight control of expression. In this vector, the recombinant protein is typically fused to a modified thioredoxin carrying several histidine residues which enable purification by nickel chromatography. The recombinant protein can be cleaved from the thioredoxin moiety by enterokinase enzyme.

Affinity chromatography may also be used with fixed antibodies or some other agent which strongly binds the peptide of interest to purify the protein from the native organism.

Purified immunogenic peptides may be used in combination with other C. difficile sub-units as a combined vaccine against C. difficile. Potential candidates are the products of the other sip genes, which share limited homology with the slpA gene product and with the N-acetylmuramoyl L-alanine amidase, (CwlB), from Bacillus subtilis, and which may be involved in remodelling of the peptidoglycan.

Other purified proteins of C. difficile to which constitutive antibodies are detected in individuals recovering from C. difficile infection are also within the scope of the present invention

A deposit of Clostridium difficile strain 171500, PCR type 1, was made at the NCIMB on Jan. 29, 2001, and accorded the accession number NCIMB 41081.

A deposit of Clostridium difficile strain 170324, PCR type 12, was made at the NCIMB on Jan. 29, 2001, and accorded the accession number NCIMB 41080.

Two peptides of the invention were found to contain the following sequences:

| | |
|---|---|
| 33kDa peptide | |
| SEQ ID No. 1: | DKTKVETADQGYTVVQSKYK |
| 31kDa peptide | |
| SEQ ID No. 2 | ATTGTQGYTVVKNDGKKAVK |

The invention will be more clearly understood from the following examples.

### EXAMPLE 1

### Clinical Study

Examination of sequential antibody responses to C. difficile among elderly patients who developed the disease was carried out. The study was based on the hypothesis that the host immune response influenced the development of Clostridium difficile disease. In particular we determined that a particular pattern of immune response to C. difficile antigens correlated with the outcome of CDD.

Materials and Methods

Patients

Serum was collected from over 300 patients and of these 30 patients developed CDD. The infecting strain (homologous strain) was grown from each patient. Strains of C. difficile were typed at the Anaerobe Reference Laboratory, Wales [O'Neill et al., 1996]. The most common strains isolated were PCR type 1 (n=15) which is the most common type causing epidemics and PCR type 12 (n=5) which is also a common hospital strain. Pre-infection serum samples were obtained from patients. Acute phase sera were then collected from patients who developed C. difficile disease. Convalescent sera were collected from patients who recovered. Protein extracts of patients' infecting C. difficile strain were probed with the patients sera using Western blotting. IgG responses to the antigens were examined.

Western Blotting

Proteins from SDS-PAGE gels were electroblotted (0.8 mA/cm2 for 1 h) to PVDF membrane using a semi-dry blotting apparatus (Atto). Primary antibodies (human serum: 1/50-1/10,000 dilution) were detected using a 1/5000 dilution of anti-human IgG (horse radish peroxidase-conjugated) in combination with enhanced chemiluminesence (ECL). Blots were washed in phosphate buffered saline (pH 7.5) containing Tween 20 (0.1% v/v), and incubated in the same solution comprising dried skim milk (5% w/v) and antibodies at the appropriate concentration. Blots were exposed to Kodak X-OMAT film for various periods of time and developed.

Results

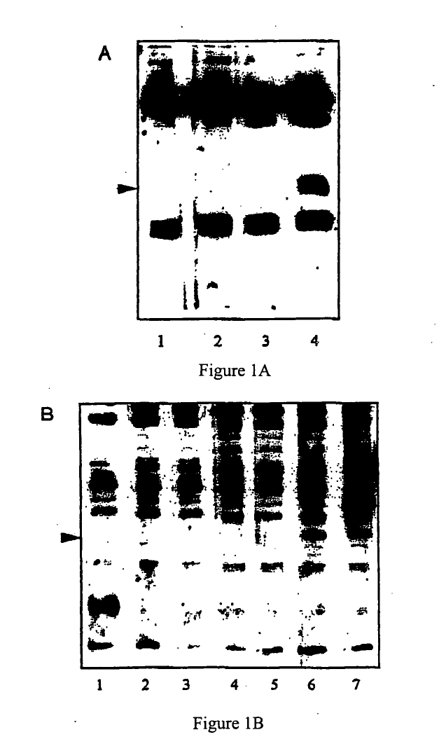

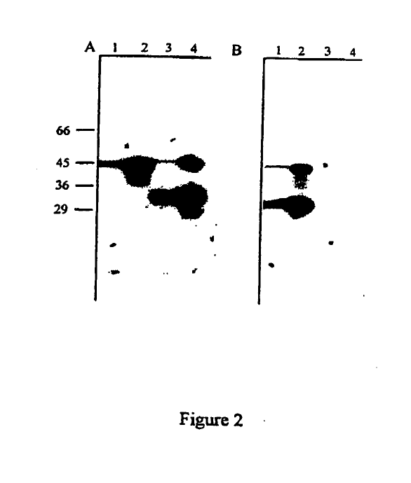

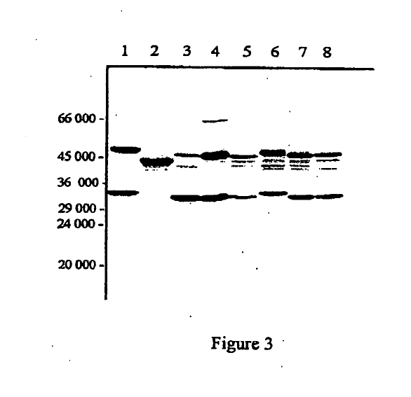

Overall 5 patients made a full recovery and new antibody responses to previously unrecognised antigens were evident in 4 of these patients. Three of these patients had C. difficile belonging to PCR type I and one patient had C. difficile PCR type 12. These patients developed an acute phase antibody response to previously unrecognised C. difficile antigens which persisted during convalescence (FIGS. 1A and 1B). These antigens were recognised by antibodies from patients who recovered and represent potential candidate vaccine antigens. FIG. 1A shows a strong reaction of convalescent antibodies was observed with the 33 kDa antigen (Lane 4, arrow). FIG. 1B shows a strong reaction of convalescent antibodies was observed with the 31 kDa antigen (Lanes 6 and 7, arrow).

These antibody responses have also been found in some controls in the same ward who were also on antibiotics but who did not develop CDD.

### EXAMPLE 2

### Further Characterisation of Protective Antigens

Materials and Methods

Partial purification and N-terminal sequencing of the 33 kDa and the 31 kDa proteins The antigens were partially purified from C. difficile based on their molecular weight using preparative continuous-elution SDS-PAGE on a model 491 Prep-Cell (Bio-Rad). The appropriate antigens were subsequently identified on Western blots probed with serum obtained from individuals who recovered from C. difficile infection.

Preparation of Surface Layer Proteins (SLPs)

SLPs were purified from C. difficile by extracting washed cells with 8 M urea, in 50 mM Tris HCl, pH 8.3 in the presence of a cocktail of protease inhibitors (Complete®, Boehringer Mannheim), for 1 h at 37° C., followed by centrifugation for 19 000×g for 30 min. The SLPs were recovered in the supernatant and dialysed to remove the urea [Cerquetti et al., 2000].

Results

The immunodominant protein which was associated with a positive outcome from C. difficile strain 171500 (PCR type 1) was identified and purified using preparative SDS-PAGE. The N-terminal region of the protein was sequenced using an Applied Biosystems Procise Sequencer, viz DKTKVETADQGYTVVQSKYK (SEQ ID No. 1)

The antigen which was associated with a protective antibody response from the C. difficile strain 170324 (PCR type 12) was identified and the N-terminal sequence obtained, viz ATTGTQGYTVVKNDGKKAVK (SEQ ID No. 2).

These sequences were used to interrogate the C. diffcile genome sequence using the TBLASTN programme, which compared our query sequences with those of the genome project (available at web address http://www.sanger.ac.uk/Projects/C_difficile/), translated in all 6 possible reading frames. A nearly identical stretch of sequence was identified when the sequence from strain 1710324 (type 12) was used for interrogation. The same stretch of sequence was picked up with the sequence from strain 171500 (type 1) was used, although the identity was much less strong. Since the homologous sequence belonged to an open reading frame encoding a 719-residue peptide, this result was somewhat surprising. However, when the N-terminal sequences from the higher molecular weight SLP component were later published by Cerquetti et al [2000], it became apparent that they were encoded downstream along the same gene, subsequently identified as slpA, and the reason for the discrepancy in size between the gene and its products became readily apparent.

The purified SLPs from strains 171500 (PCR type 1) and 170324 (PCR type 12) showed strong reactivity with homologous convalescent serum, and co-migrated with the dominant antigens detected in crude cell extracts as shown in FIG. 2. Lanes 1 and 3 contain crude antigen preparations from PCR types 1 and 12 respectively, and Lanes 2 and 4 contain SLP preparations from PCR types 1 and 12, respectively. Panel A was probed with serum from a patient recovering from infection with PCR type 1, and Panel B was probed with serum from a patient recovering from infection with PCR type 12. Each serum detected 2 major antigens in the infecting strain (Panel A, Lane 3); (Panel B, Lane 1), which co-migrated with the 2 SLPs (Panel A, Lane 4; Panel B, Lane 2), with which the sera also reacted strongly. Note that serum from the patient infected with the PCR type 1 strain recognised the higher molecular weight SLP from the PCR type 12 strain (Panel A, Lanes 1 and 2), whereas the converse did not occur (Panel B, Lanes 3 and 4). There is no apparent antigenic cross-reactivity with regard to the lower molecular weight SLPs.

SLPs were prepared from selected strains by urea extraction, and subjected to SDS-PAGE and staining with Coomassie Blue (FIG. 3). Most strains showed a characteristic profile, with two major bands located in the 29 000 to 36 000 and 45 000 to 50 000 molecular weight range. An exception was strain 172450 (FIG. 3, Lane 2), which showed a single, high molecular weight band, approximately 43 000 in size.

Cloning, Sequencing and Analysis of slpA Genes

The nucleotide sequences of the slpA genes from the two sample strains of C. difficile (PCR types 1 and 12, deposited at the NCIMB) and of several others (PCR types 5, 12, 17, 31, 46 and 92, available from the Anaerobe Reference Unit at the Department of Medical Microbiology and Public Health Laboratory, Cardiff, Wales were obtained. The slpA gene and flanking sequence was amplified by polymerase chain reaction from genomic DNA prepared from C. difficile using a commercial kit (Puregene® DNA isolation kit for yeast and Gram positive bacteria, Gentra systems Minneapolis, Minn.). The forward primer (5′ ATGGATTATTATAGAGATGTGAG 3′), was based on sequence from the genome sequencing project, starting 112 nucleotides upstream from the start of the slpA open reading frame. Two reverse primers were used, depending on the PCR type. A downstream primer (5′ CTATTTAAAGTTTTATTAAAACTTATATTAC 3′) was used to amplify slpA from PCR types 12, 17, 31, 46 and 92. A reverse primer based on the 3′ end of the slpA open reading frame from strain 630 and the subsequent nonsense codon (5′ TTACATATCTAATAAATCTTTCATTTTGTTTATAACTG 3′) was used to amplify slpA from PCR types 1 and 5. The choice of primer for the latter two PCR types may have resulted in a small number of systematic errors in the nucleotide sequence obtained. PCR was carried out using HotStar™ Taq polymerase (Qiagen Ltd., Crawley, West Sussex, UK) according to the manufacturer's instructions. A single fragment of approximately 2 kb was obtained for each strain, which was then cloned into the pBAD/Thio TOPO vector (Invitrogen, Groningen, Netherlands). Inserts were sequenced from both ends by standard procedures in commercial facilities at MWG (Wolverton Mill South, Milton Keynes, UK) and Cambridge University. New primers were designed on the basis of initial sequencing results, enabling sequencing of both strands to be completed (a process known as chromosome walking).

The results are shown in Appendices 1-8.

The nucleotide sequences were translated to enable prediction of the amino acid sequence(s) of the product(s) (Appendices 1-8). The N-terminal sequences obtained experimentally for the low molecular weight protective antigens from strains 171500 (PCR type 1) and 170324 (PCR type 12) were almost identical to those predicted from the nucleotide sequences of their respective slpA genes (18/20 identical residues for strain 171500, and 19/20 identical residues for strain 170324).

Appendix 1 shows the open reading frame with translation for slpA from strain 171500 (PCR type 1), SEQ ID No 3. Since the reverse primer was based on the 35 nucleotides from the 3′ end of the s/pa gene, the sequence is not necessarily 100% accurate in this region. However, this part of the gene does not seem to vary greatly from strain to strain.

Appendix 2 shows the open reading frame with translation for slpA from strain 172450 (PCR type 5), SEQ ID No 4. Again, the sequence obtained for the 3′ 35 nucleotides is not fully reliable. This gene is considerably smaller than the other slpA genes sequenced, and shows strong sequence divergence from the other PCR types examined.

Appendix 3 shows the open reading frame with translation for slpA from strain 170324 (PCR type 12), SEQ ID No 5. This gene showed a single base difference when compared with the strain used for the genome sequencing project, strain 630, of the same PCR type. The deduced amino acid sequence is identical.

Appendix 4 shows the open reading frame with translation for slpA from strain 171448 (PCR type 12), SEQ ID No 6. This gene was almost identical in sequence to that from strain 170324.

Appendix 5 shows the open reading frame with translation for slpA from strain 171862 (PCR type 17), SEQ ID No 7.

Appendix 6 shows the open reading frame with translation for slpA from strain 173644 (PCR type 31), SEQ ID No 8. Like the slpA from strain 172450, this sequence is very dissimilar to those of slpA genes from other PCR types encountered.

Appendix 7 shows the open reading frame with translation for slpA from strain 170444 (PCR type 46), SEQ ID No 9. This sequence is virtually identical to that obtained for slpA from PCR type 12 and 92 strains.

Appendix 8 shows the open reading frame with translation for slpA from strain 170426 (PCR type 92), SEQ ID No 10. This sequence is virtually identical to that obtained for slpA from PCR type 12 and 46.

The cleavage site of the putative signal sequences from both genes was determined from experimental evidence (the N-terminal sequence of the mature proteins as determined by Edman degradation), and by the prediction tool of the Centre for Biological Sequence Analysis at the Technical University of Denmark [Nielsen et al., 1997]. The site for cleavage of the slpA gene product to form the mature SLPs was predicted from experimental [Cerquetti et al., 2000, Karjalainen et al., 2001 and Calabi et al., 2001]. The cleavage site is typically preceded by the motif TKS. However, the relevant motif is likely to be TKG in strain 173644 (PCR type 31). No obvious motif appeared for strain 172450 (PCR type 5). However, the protein produced by type 5 strains does appear to be cleaved; hence we predicted the site to occur at a point where the SLP sequence aligns with the cleavage sites of other PCR types.

The molecular weight and isoelectric point was calculated for each of the predicted mature proteins by the ExPASy server of the Swiss Institute for Bioinformatics (Table 1). In general, the calculated molecular weights were in fair agreement with apparent molecular masses determined from migration in gels (FIG. 3). No lower molecular weight band was apparent for Strain 172450 (PCR type 5; Lane 2). However, a higher molecular weight band is present, which is similar in size to the predicted weight for the C-terminal moiety. We observed a similar profile for another type 5 strain. It is possible that the lower molecular weight species is subject to degradation in this strain. Another possibility is that it is heavily glycosylated, which can affect staining. All peptides had a predicted isoelectric point below 7, typical of acidic proteins, and characteristic of SLPs in general [Sleyter et al, 1993].

TABLE 1

| | | | | MW |
|---|---|---|---|---|
| C. difficile strain | pI | pI | MW | (C- |
| (PCR type) | (N-terminal) | (C-terminal) | (N-terminal) | terminal) |
| 171500 (Type 1) | 4.83 | 4.66 | 33365.41 | 44220.37 |
| 172450 (Type 5) | 4.86 | 4.65 | 19364.46 | 42757.63 |
| 170324 (Type 12) | 4.92 | 4.58 | 34228.25 | 39522.24 |
| 171448 (Type 12) | 4.98 | 4.58 | 34156.18 | 39492.21 |
| 171862 (Type 17) | 5.09 | 4.53 | 33783.73 | 39407.11 |
| 173644 (Type 31) | 5.05 | 4.56 | 33626.48 | 41821.69 |
| 170444 (Type 46) | 5.06 | 4.58 | 34230.31 | 39522.24 |
| 170426 (Type 92) | 4.99 | 4.58 | 34242.32 | 39522.24 |

The translated nucleotide sequences were compared with published SlpA sequences (EMBL Accession numbers AJ300676, and AJ300677 for examples from PCR types 1, and 17 respectively; strain 630 available from the Sanger Institute for PCR type 12; EMBL Accession number AY004256 for a variant from an unnamed PCR type). The Clustal W alignment programme, which is freely available, was used. Where SlpA sequences from our isolates were compared with those of other strains of the same PCR types, they were found to be nearly or quite identical. This observation indicates, together with existing knowledge from serotyping, that the number of variants of slpA is not infinite, and that natural evolution of the gene is not rapid. Table 2 shows a compilation of homologies, based on amino acid residue identity, for the different translated sequences measured against published sequences. Homologies are compiled for the predicted mature peptides, either combined (Table 2A) or as N-terminal (low molecular weight, less conserved moiety) (Table 2B) and C-terminal (high molecular weight, more conserved) (Table 2C) mature peptides according to predicted cleavage sites. It is clear that the SlpA sequences from strains 172450 (PCR type 5) and 173644 (PCR type 31) are quite distinct particularly with respect to N-terminal region.

TABLE 2A

| | 630 | AJ300676 | AJ300677 | AY004256 |
|---|---|---|---|---|
| Strain.type | (type 12) | (type 1) | (type 17) | (type unknown) |
| 171500.type1 | 55.2 | 99.7 | 55.4 | 56.42 |
| 172450.type5 | 49.8 | 54.0 | 49.9 | 47.77 |
| 170324.type12 | 100.0 | 57.8 | 81.7 | 59.77 |
| 171448.type12 | 99.7 | | | |
| 171862.type17 | 82.3 | 58.7 | 100 | 57.54 |
| 173644.type31 | 57.9 | 59.2 | 60.1 | 56.88 |
| 170444.type46 | 99.6 | | | |
| 170426.type92 | 99.9 | | | |

TABLE 2B

| | 630 | AJ300676 | AJ300677 | AY004256 |
|---|---|---|---|---|
| Strain.type | (type 12) | (type 1) | (type 17) | (type unknown) |
| 171500.type1 | 35.4 | 100 | 34.5 | 33.54 |
| 172450.type5 | 31.6 | 32.2 | 31.0 | 24.58 |
| 170324.type12 | 100 | 34.9 | 64.6 | 36.14 |
| 171448.type12 | 99.7 | | | |
| 171862.type17 | 64.3 | 34.4 | 100 | 31.55 |
| 173644.type31 | 37.5 | 34.1 | 41.3 | 31.86 |
| 170444.type46 | 99.1 | | | |
| 170426.type92 | 99.7 | | | |

TABLE 2C

| | 630 | AJ300676 | AJ300677 | AY004256 |
|---|---|---|---|---|
| Strain.type | (type 12) | (type 1) | (type 17) | (type unknown) |
| 171500.type1 | 70.2 | 99.5 | 71.2 | 73.80 |
| 172450.type5 | 58.4 | 60.4 | 63.0 | 57.60 |
| 170324.type12 | 100 | 77.3 | 97.1 | 80.00 |
| 171448.type12 | 99.7 | | | |
| 171862.type17 | 97.3 | 78.8 | 100 | 79.62 |
| 173644.type31 | 74.1 | 78.9 | 75.1 | 75.38 |
| 170444.type46 | 100 | | | |
| 170426.type92 | 100 | | | |

The term antibody used throughout the specification includes but is not limited to polyclonal, monoclonal, chimeric, single chain, Fab fragments and fragments produced by a Fab expression library.

The antibodies and fragments thereof may be humanised antibodies. Neutralising antibodies such as those which inhibit biological activity of the substance amino acid sequence are especially preferred for diagnostics and therapeutics.

Antibodies both polyclonal and monoclonal which are directed against epitopes obtainable from a polypeptide or peptide of the present invention are particularly useful in diagnosis and those which are neutralising are useful in passive immunotherapy.

Antibodies may be produced by any of the standard techniques well known in the art.

A therapeutically effective amount of the polypeptide, polynucleotide, peptide or antibody of the invention in the form of pharmaceutical composition may be administered. The composition may optionally comprise a pharmaceutically acceptable carrier, diluent or excipients and including combinations thereof. The pharmaceutical composition may be used in conjugation with one or more additional pharmaceutically active compounds and/or adjuvants.

Different adjuvants depending on the host may be used to increase immunological response. The adjuvant may be selected from the group comprising Freunds, mineral gels such as aluminium hydroxide and surface active substances.

The vaccine of the invention may be in the form of an immune modulating composition or pharmaceutical composition and may be administered by a number of different routes such as by injection (which includes parenteral, subcutaneous and intramuscular injection) intranasal, intramuscular, mucosal, oral, intra-vaginal, urethral or ocular administration. There may be different formulation/composition requirements dependent on the different delivery systems.

The invention is not limited to the embodiments hereinbefore described which may be varied in detail.

### REFERENCES

Calabi E., Ward S., Wren B., Paxton T., Panico M., Morris H., Dell A., Dougan G., Fairweather N. (2001). Molecular characterization of the surface layer proteins from Clostridium difficile. Mol. Microbiol. 40:1187-1199.
Cerquetti M., Molinari A., Sebastianelli A., Diociaiuti M., Petruzzelli R., Cap C., Mastrantonio P. (2000). Characterization of surface layer proteins from different Clostridium difficile clinical isolates. Microbial Pathogenesis, 28:363-372.
Cheng S. H, Lu J. J, Young T. G, Perng C. L, Chi W. M. (1997) Clostridium difficile-associated diseases: comparison of symptomatic infection versus carriage on the basis of risk factors, toxin production, and genotyping results. Clin Infect Dis; 25: 157-8.
Delmée M., Laroche Y., Avesani V., Cornelis G. (1986). Comparison of serogrouping and polyacrylamide gel electrophoresis for typing Clostridium difficile. J. Clin. Microbiol. 24:991-994.
Delmée M., Avesani V., Delferrière N., Burtonboy G. (1990). Characterization of flagella of Clostridium difficile and their role in serogrouping reactions.
Karjalainen T., Waligora-Dupriet A.-J., Cerquetti M., Spigaglia P., Maggioni A., Mauri P., Mastrantonio P., (2001). Molecular and genomic analysis of genes encoding surface-anchored proteins from Clostridium difficile. Infect. Immun. 69:3442-3446.
Kawata T., Takeoka A., Takumi K., Masuda K. (1984). Demonstration and preliminary characterization of a regular array in the cell wall of Clostridium difficile. FEMS Microbiol. Lett 24:323-328.
Kelly, C. P., Pothoulakis C and LaMont J. T. Clostridium difficile colitis. New England Journal of Medicine. 1994 330: 257-262.
Kyne L, Warny M, Qamar A, Kelly C. Asymptomtic carriage of Clostridium difficile and serum levels of IgG antibody against Toxin A. New England Journal of Medicine 2000; 390-7.
Leung Y. M, Kelly C. P, Boguniewicz M, Pothoulakis C, LaMont J. T, Flores A. Treatment with intravenous gamma globulin of chronic relapsing colitis by Clostridium difficile; toxin: J. Pediatr 1991; 118: 633-7.
McFarland L. V, Elmer G. W, Stamm W. E, Mulligan M. E. Correlation of immunoblot type, enterotoxin production, and cytokine production with clinical manifestation of Clostridium difficile infection in a cohort of hospitalised patients. Infect Immnun. 1991; 59: 2456-62.
Mulligan M. E, Miller S. D, McFarland L. V, Fung H. C, Kwok R. Y. Elevated levels of serum immunoglobulins in asymptomatic carriers of Clostridium difficile. Clin Infect Dis 1993: 16(Suppl 4); S239-44.
Nielsen H., Engelbrecht J., Brunak S., von Heijne G. (1997). Identification of prokaryotic and eukaryotic signal peptides and prediction of their cleavage sites. Protein Eng. 10:1-6.
O'Neill G. L., Ogunsota F. T, Brazier J. S, Duerdon B. I, Modification of a PCR ribotyping method for application as a routine typing scheme for Clostridium difficile. Anaerobe (1996) 2, 205-209.
Pantosti A, Cerquetti M, Viti F, Ortisi G, Mastratonio P. Immunoblot Of Serum Immunoglobulin G Response to Surface Proteins of Clostridium difficile in Patients With Antibiotic Associated Diarrhoea. J. Clin Microbiol 1989: 27; 2594-7.
Pelmutter D. H, Leichtnr A. M, Goldman H, Winter H. S. Chronic diarrahoea associated with hypogammaglobulinaemia and enteropathy in infants and children: Dig Dis Sci 1985; 30; 1149-55.
Poxton I. R., Higgins P. G., Currie C. G., McCoubrey J. (1999). Variation in the cell surface proteins of Clostridium difficile. Anaerobe 5:213-215.
Shim J. Johnson S, Samone M, Bliss D Z, Gerding D. N. Primary symptomless colonisation by Clostridium difficile and decreased risk of subsequent diarrhoea. The Lancet Vol 351 1998: 633-5.

Sleytr U. B., Messner P., Pum D., Sára M. (1993). Crystalline bacterial cell surface layers. Mol. Microbiol. 10:911-916.

APPENDIX 1

SEQ ID No. 3. Nucleotide sequence of slpA from Clostridium
difficile strain 171500, PCR type 1, with translation. The
putative secretory signal cleavage site (□) and site of
cleavage to form the two mature SLPs (♦) are indicated.

| | | |
|---|---|---|
| 1 | ATGAATAAGAAAAATATAGCAATAGCTATGTCAGGTTTAACAGTTTTAGCTTCGGCTGCA | 60 |
| | ---------+---------+---------+---------+---------+--------- | |
| 1 | M N K K N I A I A M S G L T V L A S A A | 20 |
| 61 | CCTGTATTTGCAGATGATACAAAAGTTGAAACTGGTGATCAAGGATATACAGTGGTACAA | 120 |
| | ---------+---------+---------+---------+---------+--------- | |
| 21 | P V F A D D T K V E T G D Q G Y T V V Q | 40 |
| | □ | |
| 121 | AGCAAGTATAAGAAAGCTGTTGAACAATTACAAAAAGGAATATTAGATGGAAGTATAACA | 180 |
| | ---------+---------+---------+---------+---------+--------- | |
| 41 | S K Y K K A V E Q L Q K G I L D G S I T | 60 |
| 181 | GAAATTAAAGTTTTCTTTGAGGGAACTTTAGCATCTACTATAAAAGTAGGTTCTGAGCTT | 240 |
| | ---------+---------+---------+---------+---------+--------- | |
| 61 | E I K V F F E G T L A S T I K V G S E L | 80 |
| 241 | AATGCAGCAGATGCAAGTAAATTATTGTTTACACAAGTAGATAATAAACTAGATAATTTA | 300 |
| | ---------+---------+---------+---------+---------+--------- | |
| 81 | N A A D A S K L L F T Q V D N K L D N L | 100 |
| 301 | GGTGATGGAGATTATGTAGATTTCTTAATAACTTCTCCAGGTCAAGGGGATAAAATAACT | 360 |
| | ---------+---------+---------+---------+---------+--------- | |
| 101 | G D G D Y V D F L I T S P G Q G D K I T | 120 |
| 361 | ACAAGTAAACTTGTTGCATTGAAAGATTTAACAGGTGCTTCAGCAGATGCTATAATTGCT | 420 |
| | ---------+---------+---------+---------+---------+--------- | |
| 121 | T S K L V A L K D L T G A S A D A I I A | 140 |
| 421 | GGAACATCTTCAGCAGATGGTGTTGTTACAAATACTGGAGCTGCTAGTGGTTCTACTGAG | 480 |
| | ---------+---------+---------+---------+---------+--------- | |
| 141 | G T S S A D G V V T N T G A A S G S T E | 160 |
| 481 | ACAAATTCAGCAGGAACAAAACTTGCAATGTCAGCTATTTTTGACACAGCATATACAGAT | 540 |
| | ---------+---------+---------+---------+---------+--------- | |
| 161 | T N S A G T K L A M S A I F D T A Y T D | 180 |
| 541 | TCATCTGAAACTGCGGTTAAGATTACTATAAAAGCAGATATGAATGATACTAAATTTGGT | 600 |
| | ---------+---------+---------+---------+---------+--------- | |
| 181 | S S E T A V K I T I K A D M N D T K F G | 200 |
| 601 | AAAGCAGGTGAGACAACTTATTCAACTGGGCTTACATTTGAAGATGGGTCTACAGAAAAA | 660 |
| | ---------+---------+---------+---------+---------+--------- | |
| 201 | K A G E T T Y S T G L T F E D G S T E K | 220 |
| 661 | ATTGTTAAATTAGGGGACAGTGATATTATAGATATAACTAAAGCTCTTAAACTTACTGTT | 720 |
| | ---------+---------+---------+---------+---------+--------- | |
| 221 | I V K L G D S D I I D I T K A L K L T V | 240 |
| 721 | GTTCCTGGAAGTAAAGCAACTGTTAAGTTTGCTGAAAAAACACCAAGTGCCAGTGTTCAA | 780 |
| | ---------+---------+---------+---------+---------+--------- | |
| 241 | V P G S K A T V K F A E K T P S A S V Q | 260 |
| 781 | CCAGTAATAACAAAGCTTAGAATAATAAATGCTAAAGAAGAAACAATAGATATTGACGCT | 840 |
| | ---------+---------+---------+---------+---------+--------- | |
| 261 | P V I T K L R I I N A K E E T I D I D A | 280 |
| 841 | AGTTCTAGTAAAACAGCACAAGATTTAGCTAAAAAATATGTATTTAATAAAACTGATTTA | 900 |
| | ---------+---------+---------+---------+---------+--------- | |
| 281 | S S S K T A Q D L A K K Y V F N K T D L | 300 |
| 901 | AATACTCTTTATAAAGTATTAAATGGAGATGAAGCAGATACTAATGGATTAATAGAAGAA | 960 |
| | ---------+---------+---------+---------+---------+--------- | |
| 301 | N T L Y K V L N G D E A D T N G L I E E | 320 |
| 961 | GTTAGTGGAAAATATCAAGTAGTTCTTTATCCAGAAGGAAAAAGAGTTACAACTAAGAGT | 1020 |
| | ---------+---------+---------+---------+---------+--------- | |
| 321 | V S G K Y Q V V L Y P E G K R V T T K S | 340 |
| 1021 | GCTGCAAAGGCTTCAATTGCTGATGAAAATTCACCAGTTAAATTAACTCTTAAGTCAGAT | 1080 |
| | ---------+---------+---------+---------+---------+--------- | |
| 341 | A A K A S I A D E N S P V K L T L K S D | 360 |
| | ♦ | |
| 1081 | AAGAAGAAAGACTTAAAAGATTATGTGGATGATTTAAGAACATATAATAATGGATATTCA | 1140 |
| | ---------+---------+---------+---------+---------+--------- | |
| 361 | K K K D L K D Y V D D L R T Y N N G Y S | 380 |
| 1141 | AATGCTATAGAAGTAGCAGGAGAAGATAGAATAGAAACTGCAATAGCATTAAGTCAAAAA | 1200 |
| | ---------+---------+---------+---------+---------+--------- | |
| 381 | N A I E V A G E D R I E T A I A L S Q K | 400 |
| 1201 | TATTATAACTCTGATGATGAAAATGCTATATTTAGAGATTCAGTTGATAATGTAGTATTG | 1260 |
| | ---------+---------+---------+---------+---------+--------- | |
| 401 | Y Y N S D D E N A I F R D S V D N V V L | 420 |
| 1261 | GTTGGAGGAAATGCAATAGTTGATGGACTTGTAGCTTCTCCTTTAGCTTCTGAAAAGAAA | 1320 |
| | ---------+---------+---------+---------+---------+--------- | |
| 421 | V G G N A I V D G L V A S P L A S E K K | 440 |
| 1321 | GCTCCTTTATTATTAACTTCAAAAGATAAATTAGATTCAAGCGTAAAAGCTGAAATAAAG | 1380 |
| | ---------+---------+---------+---------+---------+--------- | |
| 441 | A P L L L T S K D K L D S S V K A E I K | 460 |
| 1381 | AGAGTTATGAATATAAAGAGTACAACAGGTATAAATACTTCAAAGAAAGTTTATTTAGCT | 1440 |
| | ---------+---------+---------+---------+---------+--------- | |
| 461 | R V M N I K S T T G I N T S K K V Y L A | 480 |
| 1441 | GGTGGAGTTAATTCTATATCTAAAGAAGTAGAAAATGAATTAAAAGATATGGGACTTAAA | 1500 |
| | ---------+---------+---------+---------+---------+--------- | |
| 481 | G G V N S I S K E V E N E L K D M G L K | 500 |
| 1501 | GTTACAAGATTAGCAGGAGATGATAGATATGAAACTTCTCTAAAAATAGCTGATGAAGTA | 1560 |
| | ---------+---------+---------+---------+---------+--------- | |
| 501 | V T R L A G D D R Y E T S L K I A D E V | 520 |
| 1561 | GGTCTTGATAATGATAAAGCATTTGTAGTTGGAGGAACAGGATTAGCAGATGCCATGAGT | 1620 |
| | ---------+---------+---------+---------+---------+--------- | |
| 521 | G L D N D K A F V V G G T G L A D A M S | 540 |
| 1621 | ATAGCTCCAGTTGCATCTCAATTAAGAAATGCTAATGGTAAAATGGATTTAGCTGATGGT | 1680 |
| | ---------+---------+---------+---------+---------+--------- | |
| 541 | I A P V A S Q L R N A N G K N D L A D G | 560 |
| 1681 | GATGCTACACCAATAGTAGTTGTAGATGGAAAAGCTAAAACTATAAATGATGATGTAAAA | 1740 |
| | ---------+---------+---------+---------+---------+--------- | |
| 561 | D A T P I V V V D G K A K T I N D D V K | 580 |
| 1741 | GATTTCTTAGATGATTCACAAGTTGATATAATAGGTGGAGAAAACAGTGTATCTAAAGAT | 1800 |
| | ---------+---------+---------+---------+---------+--------- | |
| 581 | D F L D D S Q V D I I G G E N S V S K D | 600 |
| 1801 | GTTGAAAATGCAATAGATGATGCTACAGGTAAATCTCCAGATAGATATAGTGGAGATGAT | 1860 |
| | ---------+---------+---------+---------+---------+--------- | |
| 601 | V E N A I D D A T G K S P D R Y S G D D | 620 |
| 1861 | AGACAAGCAACTAATGCAAAAGTTATAAAAGAATCTTCTTATTATCAAGATAACTTAAAT | 1920 |
| | ---------+---------+---------+---------+---------+--------- | |
| 621 | R Q A T N A K V I K E S S Y Y Q D N L N | 640 |
| 1921 | AATGATAAAAAAGTAGTTAATTTCTTTGTAGCTAAAGATGGTTCTACTAAAGAAGATCAA | 1980 |
| | ---------+---------+---------+---------+---------+--------- | |
| 641 | N D K K V V N F F V A K D G S T K E D Q | 660 |
| 1981 | TTAGTTGATGCTTTAGCAGCAGCTCCAGTTGCAGCAAACTTTGGTGTAACTCTTAATTCT | 2040 |
| | ---------+---------+---------+---------+---------+--------- | |
| 661 | L V D A L A A A P V A A N F G V T L N S | 680 |
| 2041 | GATGGTAAGCCAGTAGATAAAGATGGTAAAGtATTAACTGGTTCTGATAATGATAAAAAT | 2100 |
| | ---------+---------+---------+---------+---------+--------- | |
| 681 | D G K P V D K D G K V L T G S D N D K N | 700 |
| 2101 | AAATTAGTATCTCCAGCACCTATAGTATTAGCTACTGATTCTTTATCTTCAGATCaAAGT | 2160 |
| | ---------+---------+---------+---------+---------+--------- | |
| 701 | K L V S P A P I V L A T D S L S S D Q S | 720 |
| 2161 | GTATCTATAAGTAaAGTTCTTGATAAAGATAATGGAGAAAACTTAGTTCAAGTTGGTAAA | 2220 |
| | ---------+---------+---------+---------+---------+--------- | |
| 721 | V S I S K V L D K D N G E N L V Q V G K | 740 |
| 2221 | GGTATAGCTACTTCAGTTATAAACAAAATGAAAGATTTATTAGATATG | 2268 |
| | ---------+---------+---------+---------+-------- | |
| 741 | G I A T S V I N K M K D L L D M | 756 |

APPENDIX 2

SEQ ID No. 4. Nucleotide sequence of slpA from Clostridium
difficile strain 172450, PCR type 5, with translation. The
putative secretory signal cleavage site (□) is indicated,
and an approximation of the and site of cleavage to form
the two mature SLPs (♦) is also indicated.

| | | |
|---|---|---|
| 1 | ATGAAAAAAAGAAATTTAGCAATGGCTATGGCAGCTGTTACTGTAGTAGGTTCTGCTGCT | 60 |
| | ---------+---------+---------+---------+---------+--------- | |
| 1 | M K K R N L A M A M A A V T V V G S A A | 20 |
| 61 | CCAGTTTTTGCAGCAGCTTCAGATGTAATATCACTACAAGATGGTACAAATGATAAGTAT | 120 |
| | ---------+---------+---------+---------+---------+--------- | |
| 21 | P V F A A A S D V I S L Q D G T N D K Y | 40 |
| | □ | |
| 121 | ACAGTATCAAATACTAAAGCTAGTGACTTAGTAAAGGATATTTTAGCAGCACAAAACTTA | 180 |
| | ---------+---------+---------+---------+---------+--------- | |
| 41 | T V S N T K A S D L V K D I L A A Q N L | 60 |
| 181 | ACAACAGGTGCAGTTATTTTGAACAAAGATACAAAAGTTACTTTCTATGATGCAAATGAG | 240 |
| | ---------+---------+---------+---------+---------+--------- | |
| 61 | T T G A V I L N K D T K V T F Y D A N E | 80 |
| 241 | AAAGATTCTTCAACTCCAACTGGAGATAAAAAAGTTTATTCAGAACAAACTTTAACTACA | 300 |
| | ---------+---------+---------+---------+---------+--------- | |
| 81 | K D S S T P T G D K K V Y S E Q T L T T | 100 |
| 301 | GCTAATGGAAATGAAGATTATGTAAAGACAACTTTAAAAAATTTAGATGCAGGAGAATAT | 360 |
| | ---------+---------+---------+---------+---------+--------- | |
| 101 | A N G N E D Y V K T T L K N L D A G E Y | 120 |
| 361 | GCTATTATAGATTTAACTTATAATAATGCTAAAACTGTTGAAATTAAAGTAGTAGCAGCT | 420 |
| | ---------+---------+---------+---------+---------+--------- | |
| 121 | A I I D L T Y N N A K T V E I K V V A A | 140 |
| 421 | AGTGAAAAAACAGTAGTTGTATCTAGTGATGCGAAAAATAGTGCAAAAGATATAGCTGAA | 480 |
| | ---------+---------+---------+---------+---------+--------- | |
| 141 | S E K T V V V S S D A K N S A K D I A E | 160 |
| 481 | AAATATGTGTTTGAAGACAAAGACTTAGAAAATGCACTAAAAACTATAAATGCCTCAGAT | 540 |
| | ---------+---------+---------+---------+---------+--------- | |
| 161 | K Y V F E D K D L E N A L K T I N A S D | 180 |
| 541 | TTCAGTAAAACTGATAGTTACTATCAAGTAGTTCTTTATCCAAAAGGAAAGAGATTACAA | 600 |
| | ---------+---------+---------+---------+---------+--------- | |
| 181 | F S K T D S Y Y Q V V L Y P K G K R L Q | 200 |
| 601 | GGTTTCTCAACTTATAGAGCTACAAATTATAATGAAGGAACTGCATATGGTAATACACCA | 660 |
| | ---------+---------+---------+---------+---------+--------- | |
| 201 | G F S T Y R A T N Y N E G T A Y G N T P | 220 |
| | ♦ | |
| 661 | GTAATATTAACTCTAAAATCTACTAGTAAGAGTAATTTAAAGACTGCAGTAGAAGAGTTA | 720 |
| | ---------+---------+---------+---------+---------+--------- | |
| 221 | V I L T L K S T S K S N L K T A V E E L | 240 |
| 721 | CAAAAATTGAATGCTAGTTATTCTAATACTACAACTTTAGCTGGTGATGACAGAATACAA | 780 |
| | ---------+---------+---------+---------+---------+--------- | |
| 241 | Q K L N A S Y S N T T T L A G D D R I Q | 260 |
| 781 | ACAGCTATAGAGATAAGTAAAGAATATTACAATAATGATGGCGAGAAATCAGATCATTCA | 840 |
| | ---------+---------+---------+---------+---------+--------- | |
| 261 | T A I E I S K E Y Y N N D G E K S D H S | 280 |
| 841 | GCTGATGTTAAAGAGAATGTTAAAAATGTTGTATTAGTAGGTGCAAATGCACTAGTAGAT | 900 |
| | ---------+---------+---------+---------+---------+--------- | |
| 281 | A D V K E N V K N V V L V G A N A L V D | 300 |
| 901 | GGATTAGTTGCGGCTCCTTTAGCAGCAGAJAAAGATGCTCCACTATTATTAACTTCAAAA | 960 |
| | ---------+---------+---------+---------+---------+--------- | |
| 301 | G L V A A P L A A E K D A P L L L T S K | 320 |
| 961 | GATAAATTAGATTCGTCAGTAAAATCTGALATAAAGAGAGTTTTAGACTTAAAAACTTCA | 1020 |
| | ---------+---------+---------+---------+---------+--------- | |
| 321 | D K L D S S V K S E I K R V L D L K T S | 340 |
| 1021 | ACAGAAGTAACAGGAAAAACAGTTTATATAGCTGGTGGAGTTAATAGTGTATCTAAAGAA | 1080 |
| | ---------+---------+---------+---------+---------+--------- | |
| 341 | T E V T G K T V Y I A G G V N S V S K E | 360 |
| 1081 | GTTGTAACAGAATTAGAATCAATGGGATTAAAAGTTGAAAGATTCTCAGGTGATGATAGA | 1140 |
| | ---------+---------+---------+---------+---------+--------- | |
| 361 | V V T E L E S M G L K V E R F S G D D R | 380 |
| 1141 | TATGAAACTTCTTTAAAAATAGCAGGTGAAATAGGCTTAGATAATGATAAGGCTTATGTA | 1200 |
| | ---------+---------+---------+---------+---------+--------- | |
| 381 | Y E T S L K I A G E I G L D N D K A Y V | 400 |
| 1201 | GTTGGTGGAACAGGATTAGCAGATGCCATGAGTATAGCTTCAGTTGCTTCTACTAAATTA | 1260 |
| | ---------+---------+---------+---------+---------+--------- | |
| 401 | V G G T G L A D A M S I A S V A S T K L | 420 |
| 1261 | GATGGTAATGGTGTTGTAGATAGAACAAATGGACATGCTACTCCAATAGTTGTTGTAGAT | 1320 |
| | ---------+---------+---------+---------+---------+--------- | |
| 421 | D G N G V V D R T N G H A T P I V V V D | 440 |
| 1321 | GGAAAAGCTGATAAAATATCTGATGACTTAGATAGTTTCTTAGGAAGCGCTGATGTAGAT | 1380 |
| | ---------+---------+---------+---------+---------+--------- | |
| 441 | G K A D K I S D D L D S F L G S A D V D | 460 |
| 1381 | ATAATAGGTGGATTTGCAAGTGTATCTGAAAAGATGGAAGAAGCTATATCAGATGCTACT | 1440 |
| | ---------+---------+---------+---------+---------+--------- | |
| 461 | I I G G F A S V S E K M E E A I S D A T | 480 |
| 1441 | GGTAAAGGCGTTACAAGAGTTAAAGGCGACGATAGACAAGACACTAACTCTGAAGTTATA | 1500 |
| | ---------+---------+---------+---------+---------+--------- | |
| 481 | G K G V T R V K G D D R Q D T N S E V I | 500 |
| 1501 | AAAACATATTATGCTAATGATACTGAAATAGCTAAAGCTGCAGTTTTAGATAAAGATTCA | 1560 |
| | ---------+---------+---------+---------+---------+--------- | |
| 501 | K T Y Y A N D T E I A K A A V L D K D S | 520 |
| 1561 | GGTGCTTCAAGTAGTGATGCAGGAGTATTTAATTTCTATGTAGCTAAAGATGGATCTACA | 1620 |
| | ---------+---------+---------+---------+---------+--------- | |
| 521 | G A S S S D A G V F N F Y V A K D G S T | 540 |
| 1621 | AAAGAAGATCAATTAGTTGATGCATTAGCAGTAGGAGCTGTTGCTGGATATAAACTTGCT | 1680 |
| | ---------+---------+---------+---------+---------+--------- | |
| 541 | K E D Q L V D A L A V G A V A G Y K L A | 560 |
| 1681 | CCAGTTGTATTAGCTACTGATTCTTTATCTTCTGATCAATCGGTTGCTATAAGCAAAGTT | 1740 |
| | ---------+---------+---------+---------+---------+--------- | |
| 561 | P V V L A T D S L S S D Q S V A I S K V | 580 |
| 1741 | GTAGGAGAAAAATATTCTAAAGATTTAACACAAGTTGGTCAAGGAATAGCTAATTCAGTT | 1800 |
| | ---------+---------+---------+---------+---------+--------- | |
| 581 | V G E K Y S K D L T Q V G Q G I A N S V | 600 |
| 1801 | ATAAACAAAATGAAAGATTTATTAGATATG | 1830 |
| | ---------+---------+---------+ | |
| 601 | I N K M K D L L D M | 610 |

APPENDIX 3

SEQ ID No. 5. Nucleotide sequence of slpA from Clostridium
difficile strain 170324, PCR type 12, with translation. The
putative secretory signal cleavage site (□) and site of
cleavage to form the two mature SLPs (♦) are indicated.

| | | |
|---|---|---|
| 1 | ATGAATAAGAAAAATATAGCAATAGCTATGTCAGGTTTAACAGTTTTAGCTTCGGCTGCT | 60 |
| | ---------+---------+---------+---------+---------+--------- | |
| 1 | M N K K N I A I A M S G L T V L A S A A | 20 |
| 61 | CCTGTTTTTGCTGCAACTACTGGAACACAAGGTTATACTGTAGTTAAAAACGACTGGAAA | 120 |
| | ---------+---------+---------+---------+---------+--------- | |
| 21 | P V F A A T T G T Q G Y T V V K N D W K | 40 |
| | □ | |
| 121 | AAAGCAGTAAAACAATTACAAGATGGACTAAAAGATAATAGTATAGGAAAGATAACTGTA | 180 |
| | ---------+---------+---------+---------+---------+--------- | |
| 41 | K A V K Q L Q D G L K D N S I G K I T V | 60 |
| 181 | TCTTTTAATGATGGGGTTGTGGGTGAAGTAGCTCCTAAAAGTGCTAATAAGAAAGCGGAC | 240 |
| | ---------+---------+---------+---------+---------+--------- | |
| 61 | S F N D G V V G E V A P K S A N K K A D | 80 |
| 241 | AGAGATGCTGCAGCTGAGAAGTTATATAATCTTGTTAACACTCAATTAGATAAATTAGGT | 300 |
| | ---------+---------+---------+---------+---------+--------- | |
| 81 | R D A A A E K L Y N L V N T Q L D K L G | 100 |
| 301 | GATGGAGATTATGTTGATTTTTCTGTAGATTATAATTTAGAAAACAAAATAATAACTAAT | 360 |
| | ---------+---------+---------+---------+---------+--------- | |
| 101 | D G D Y V D F S V D Y N L E N K I I T N | 120 |
| 361 | CAAGCAGATGCAGAAGCAATTGTTACAAAGTTAAATTCACTTAATGAGAAAACTCTTATT | 420 |
| | ---------+---------+---------+---------+---------+--------- | |
| 121 | Q A D A E A I V T K L N S L N E K T L I | 140 |
| 421 | GATATAGCAACTAAAGATACTTTTGGAATGGTTAGTAAAACACAAGATAGTGAAGGTAAA | 480 |
| | ---------+---------+---------+---------+---------+--------- | |
| 141 | D I A T K D T F G M V S K T Q D S E G K | 160 |
| 481 | AATGTTGCTGCAACAAAGGCACTTAAAGTTAAAGATGTTGCTACATTTGGTTTGAAGTCT | 540 |
| | ---------+---------+---------+---------+---------+--------- | |
| 161 | N V A A T K A L K V K D V A T F G L K S | 180 |
| 541 | GGTGGAAGCGAAGATACTGGATATGTTGTTGAAATGAAAGCAGGAGCTGTAGAGGATAAG | 600 |
| | ---------+---------+---------+---------+---------+--------- | |
| 181 | G G S E D T G Y V V E M K A G A V E D K | 200 |
| 601 | TATGGTAAAGTTGGAGATAGTACGGCAGGTATTGCAATAAATCTTCCTAGTACTGGACTT | 660 |
| | ---------+---------+---------+---------+---------+--------- | |
| 201 | Y G K V G D S T A G I A I N L P S T G L | 220 |
| 661 | GAATATGCAGGTAAAGGAACAACAATTGATTTTAATAAAACTTTAAAAGTTGATGTAACA | 720 |
| | ---------+---------+---------+---------+---------+--------- | |
| 221 | E Y A G K G T T I D F N K T L K V D V T | 240 |
| 721 | GGTGGTTCAACACCTAGTGCTGTAGCTGTAAGTGGTTTTGTAACTAAAGATGATACTGAT | 780 |
| | ---------+---------+---------+---------+---------+--------- | |
| 241 | G G S T P S A V A V S G F V T K D D T D | 260 |
| 781 | TTAGCAAAATCAGGTACTATAAATGTAAGAGTTATAAATGCAAAAGAAGAATCAATTGAT | 840 |
| | ---------+---------+---------+---------+---------+--------- | |
| 261 | L A K S G T I N V R V I N A K E E S I D | 280 |
| 841 | ATAGATGCAAGCTCATATACATCAGCTGAAAATTTAGCTAAAAGATATGTATTTGATCCA | 900 |
| | ---------+---------+---------+---------+---------+--------- | |
| 281 | I D A S S Y T S A E N L A K R Y V F D P | 300 |
| 901 | GATGAAATTTCTGAAGCATATAAGGCAATAGTAGCATTACAAAATGATGGTATAGAGTCT | 960 |
| | ---------+---------+---------+---------+---------+--------- | |
| 301 | D E I S E A Y K A I V A L Q N D G I E S | 320 |
| 961 | AACTTAGTTCAGTTAGTTAATGGAAAATATCAAGTGATTTTTTATCCAGAAGGTAAAAGA | 1020 |
| | ---------+---------+---------+---------+---------+--------- | |
| 321 | N L V Q L V N G K Y Q V I F Y P E G K R | 340 |
| 1021 | TTAGAAACTAAATCAGCAAATGATACAATAGCTAGTCAAGATACACCAGCTAAAGTAGTT | 1080 |
| | ---------+---------+---------+---------+---------+--------- | |
| 341 | L E T K S A N D T I A S Q D T P A K V V | 360 |
| | ♦ | |
| 1081 | ATAAAAGCTAATAAATTAAAAGATTTAAAAGATTATGTAGATGATTTAAAAACATATAAT | 1140 |
| | ---------+---------+---------+---------+---------+--------- | |
| 361 | I K A N K L K D L K D Y V D D L K T Y N | 380 |
| 1141 | AATACTTATTCAAATGTTGTAACAGTAGCAGGAGAAGATAGAATAGAAACTGCTATAGAA | 1200 |
| | ---------+---------+---------+---------+---------+--------- | |
| 381 | N T Y S N V V T V A G E D R I E T A I E | 400 |
| 1201 | TTAAGTAGTAAATATTATAATTCTGATGATAAAAATGCAATAACTGATAAAGCAGTTAAT | 1260 |
| | ---------+---------+---------+---------+---------+--------- | |
| 401 | L S S K Y Y N S D D K N A I T D K A V N | 420 |
| 1261 | GATATAGTATTAGTTGGATCTACATCTATAGTTGATGGTCTTGTTGCATCACCATTAGCT | 1320 |
| | ---------+---------+---------+---------+---------+--------- | |
| 421 | D I V L V G S T S I V D G L V A S P L A | 440 |
| 1321 | TCAGAAAAAACAGCTCCATTATTATTAACTTCAAAAGATAAATTAGATTCATCAGTAAAA | 1380 |
| | ---------+---------+---------+---------+---------+--------- | |
| 441 | S E K T A P L L L T S K D K L D S S V K | 460 |
| 1381 | TCTGAAATAAAGAGAGTTATGAACTTAAAGAGTGACACTGGTATAAATACTTCTAAAAAA | 1440 |
| | ---------+---------+---------+---------+---------+--------- | |
| 461 | S E I K R V M N L K S D T G I N T S K K | 480 |
| 1441 | GTTTATTTAGCTGGTGGAGTTAATTCTATATCTAAAGATGTAGAAAATGAATTGAAAAAC | 1500 |
| | ---------+---------+---------+---------+---------+--------- | |
| 481 | V Y L A G G V N S I S K D V E N E L K N | 500 |
| 1501 | ATGGGTCTTAAAGTTACTAGATTATCAGGAGAAGACAGATACGAAACTTCTTTAGCAATA | 1560 |
| | ---------+---------+---------+---------+---------+--------- | |
| 501 | M G L K V T R L S G E D R Y E T S L A I | 520 |
| 1561 | GCTGATGAAATAGGTCTTGATAATGATAAAGCATTTGTAGTTGGTGGTACTGGATTAGCA | 1620 |
| | ---------+---------+---------+---------+---------+--------- | |
| 521 | A D E I G L D N D K A F V V G G T G L A | 540 |
| 1621 | GATGCTATGAGTATAGCTCCAGTTGCTTCTCAACTTAAAGATGGAGATGCTACTCCAATA | 1680 |
| | ---------+---------+---------+---------+---------+--------- | |
| 541 | D A M S I A P V A S Q L K D G D A T P I | 560 |
| 1681 | GTAGTTGTAGATGGAAAAGCAAAAGAAATAAGTGATGATGCTAAGAGTTTCTTAGGAACT | 1740 |
| | ---------+---------+---------+---------+---------+--------- | |
| 561 | V V V D G K A K E I S D D A K S F L G T | 580 |
| 1741 | TCTGATGTTGATATAATAGGTGGAAAAAATAGCGTATCTAAAGAGATTGAAGAGTCAATA | 1800 |
| | ---------+---------+---------+---------+---------+--------- | |
| 581 | S D V D I I G G K N S V S K E I E E S I | 600 |
| 1801 | GATAGTGCAACTGGAAAAACTCCAGATAGAATAAGTGGAGATGATAGACAAGCAACTAAT | 1860 |
| | ---------+---------+---------+---------+---------+--------- | |
| 601 | D S A T G K T P D R I S G D D R Q A T N | 620 |
| 1861 | GCTGAAGTTTTAAAAGAAGATGATTATTTCACAGATGGTGAAGTTGTGAATTACTTTGTT | 1920 |
| | ---------+---------+---------+---------+---------+--------- | |
| 621 | A E V L K E D D Y F T D G E V V N Y F V | 640 |
| 1921 | GCAAAAGATGGTTCTACTAAAGAAGATCAATTAGTAGATGCCTTAGCAGCAGCACCAATA | 1980 |
| | ---------+---------+---------+---------+---------+--------- | |
| 641 | A K D G S T K E D Q L V D A L A A A P I | 660 |
| 1981 | GCAGGTAGATTTAAGGAGTCTCCAGCTCCAATCATACTAGCTACTGATACTTTATCTTCT | 2040 |
| | ---------+---------+---------+---------+---------+--------- | |
| 661 | A G R F K E S P A P I I L A T D T L S S | 680 |
| 2041 | GACCAAAATGTAGCTGTAAGTAAAGCAGTTCCTAAAGATGGTGGAACTAACTTAGTTCAA | 2100 |
| | ---------+---------+---------+---------+---------+--------- | |
| 681 | D Q N V A V S K A V P K D G G T N L V Q | 700 |
| 2101 | GTAGGTAAAGGTATAGCTTCTTCAGTTATAAACAAAATGAAAGATTTATTAGATATG | 2157 |
| | ---------+---------+---------+---------+---------+------- | |
| 701 | V G K G I A S S V I N K M K D L L D M | 719 |

APPENDIX 4

SEQ ID No 6. Nucleotide sequence of slpA from Clostridium
difficile strain 171448, PCR type 12, with translation. The
putative secretory signal cleavage site (□) and site of
cleavage to form the two mature SLPs (♦) are indicated.

| | | |
|---|---|---|
| 1 | ATGAATAAGAAAAATATAGCAATAGCTATGTCAGGTTTAACAGTTTTAGCTTCGGCTGCT | 60 |
| | ---------+---------+---------+---------+---------+--------- | |
| 1 | M N K K N I A I A M S G L T V L A S A A | 20 |
| 61 | CCTGTTTTTGCTGCAACTACTGGAACACAAGGTTATACTGTAGTTAAAAACGACTGGAAA | 120 |
| | ---------+---------+---------+---------+---------+--------- | |
| 21 | P V F A A T T G T Q G Y T V V K N D W K | 40 |
| | □ | |
| 121 | AAAGCAGTAAAACAATTACAAGATGGACTAAAAGATAATAGTATAGGAAAGATAACTGTA | 180 |
| | ---------+---------+---------+---------+---------+--------- | |
| 41 | K A V K Q L Q D G L K D N S I G K I T V | 60 |
| 181 | TCTTTTAATGATGGGGTTGTGGGTGAAGTAGCTCCTAAAAGTGCTAATAAGAAAGCGGAC | 240 |
| | ---------+---------+---------+---------+---------+--------- | |
| 61 | S F N D G V V G E V A P K S A N K K A D | 80 |
| 241 | AGAGATGCTGCAGCTGAGAAGTTATATAATCTTGTTAACACTCAATTAGATAAATTAGGT | 300 |
| | ---------+---------+---------+---------+---------+--------- | |
| 81 | R D A A A E K L Y N L V N T Q L D K L G | 100 |
| 301 | GATGGAGATTATGTTGATTTTTCTGTAGATTATAATTTAGAAAACAAAATAATAACTAAT | 360 |
| | ---------+---------+---------+---------+---------+--------- | |
| 101 | D G D Y V D F S V D Y N L E N K I I T N | 120 |
| 361 | CAAGCAGATGCAGAAGCAATTGTTACAAAGTTAAATTCACTTAATGAGAAAACTCTTATT | 420 |
| | ---------+---------+---------+---------+---------+--------- | |
| 121 | Q A D A E A I V T K L N S L N E K T L I | 140 |
| 421 | GATATAGCAACTAAAGATACTTTTGGAATGGTTAGTAAAACACAAGATAGTGGAGGTAAA | 480 |
| | ---------+---------+---------+---------+---------+--------- | |
| 141 | D I A T K D T F G M V S K T Q D S G G K | 160 |
| 481 | AATGTTGCTGCAACAAAGGCACTTAAAGTTAAAGATGTTGCTACATTTGGTTTGAAGTCT | 540 |
| | ---------+---------+---------+---------+---------+--------- | |
| 161 | N V A A T K A L K V K D V A T F G L K S | 180 |
| 541 | GGTGGAAGCGAAGATACTGGATATGTTGTTGAAATGAAAGCAGGAGCTGTAGAGGATAAG | 600 |
| | ---------+---------+---------+---------+---------+--------- | |
| 181 | G G S E D T G Y V V E M K A G A V E D K | 200 |
| 601 | TATGGTAAAGTTGGAGATAGTACGGCAGGTATTGCAATAAATCTTCCTAGTACTGGACTT | 660 |
| | ---------+---------+---------+---------+---------+--------- | |
| 201 | Y G K V G D S T A G I A I N L P S T G L | 220 |
| 661 | GAATATGCAGGTAAAGGAACAACAATTGATTTTAATAAAACTTTAAAAGTTGATGTAACA | 720 |
| | ---------+---------+---------+---------+---------+--------- | |
| 221 | E Y A G K G T T I D F N K T L K V D V T | 240 |
| 721 | GGTGGTTCAACACCTAGTGCTGTAGCTGTAAGTGGTTTTGTAACTAAAGATGATACTGAT | 780 |
| | ---------+---------+---------+---------+---------+--------- | |
| 241 | G G S T P S A V A V S G F V T K D D T D | 260 |
| 781 | TTAGCAAAATCAGGTACTATAAATGTAAGAGTTATAAATGCAAAAGAAGAATCAATTGAT | 840 |
| | ---------+---------+---------+---------+---------+--------- | |
| 261 | L A K S G T I N V R V I N A K E E S I D | 280 |
| 841 | ATAGATGCAAGCTCATATACATCAGCTGAAAATTTAGCTAAAAGATATGTATTTGATCCA | 900 |
| | ---------+---------+---------+---------+---------+--------- | |
| 281 | I D A S S Y T S A E N L A K R Y V F D P | 300 |
| 901 | GATGAAATTTCTGAAGCATATAAGGCAATAGTAGCATTACAAAATGATGGTATAGAGTCT | 960 |
| | ---------+---------+---------+---------+---------+--------- | |
| 301 | D E I S E A Y K A I V A L Q N D G I E S | 320 |
| 961 | AATTTAGTTCAGTTAGTTAATGGAAAATATCAAGTGATTTTTTATCCAGAAGGTAAAAGA | 1020 |
| | ---------+---------+---------+---------+---------+--------- | |
| 321 | N L V Q L V N G K Y Q V I F Y P E G K R | 340 |
| 1021 | TTAGAAACTAAATCAGCAAATGATACAATAGCTAGTCAAGATACACCAGCTAAAGTAGTT | 1080 |
| | ---------+---------+---------+---------+---------+--------- | |
| 341 | L E T K S A N D T I A S Q D T P A K V V | 360 |
| | ♦ | |
| 1081 | ATAAAAGCTAATAAATTAAAAGATTTAAAAGATTATGTAGATGATTTAAAAACATATAAT | 1140 |
| | ---------+---------+---------+---------+---------+--------- | |
| 361 | I K A N K L K D L K D Y V D D L K T Y N | 380 |
| 1141 | AATACTTATTCAAATGTTGTAACAGTAGCAGGAGAAGATAGAATAGAAACTGCTATAGAA | 1200 |
| | ---------+---------+---------+---------+---------+--------- | |
| 381 | N T Y S N V V T V A G E D R I E T A I E | 400 |
| 1201 | TTAAGTAGTAAATATTATAATTCTGATGATAAAAATGCAATAACTGATAAAGCAGTTAAT | 1260 |
| | ---------+---------+---------+---------+---------+--------- | |
| 401 | L S S K Y Y N S D D K N A I T D K A V N | 420 |
| 1261 | GATATAGTATTAGTTGGATCTACATCTATAGTTGATGGTCTTGTTGCATCACCATTAGCT | 1320 |
| | ---------+---------+---------+---------+---------+--------- | |
| 421 | D I V L V G S T S I V D G L V A S P L A | 440 |
| 1321 | TCAGAAAAAACAGCTCCATTATTATTAGCTTCAAAAGATAAATTAGATTCATCAGTAAAA | 1380 |
| | ---------+---------+---------+---------+---------+--------- | |
| 441 | S E K T A P L L L A S K D K L D S S V K | 460 |
| 1381 | TCTGAAATAAAGAGAGTTATGAACTTAAAGAGTGACACTGGTATAAATACTTCTAAAAAA | 1440 |
| | ---------+---------+---------+---------+---------+--------- | |
| 461 | S E I K R V M N L K S D T G I N T S K K | 480 |
| 1441 | GTTTATTTAGCTGGTGGAGTTAATTCTATATCTAAAGATGTAGAAAATGAATTGAAAAAC | 1500 |
| | ---------+---------+---------+---------+---------+--------- | |
| 481 | V Y L A G G V N S I S K D V E N E L K N | 500 |
| 1501 | ATGGGTCTTAAAGTTACTAGATTATCAGGAGAAGACAGATACGAAACTTCTTTAGCAATA | 1560 |
| | ---------+---------+---------+---------+---------+--------- | |
| 501 | M G L K V T R L S G E D R Y E T S L A I | 520 |
| 1561 | GCTGATGAAATAGGTCTTGATAATGATAA.AGCATTTGTAGTTGGTGGTACTGGATTAGCA | 1620 |
| | ---------+---------+---------+---------+---------+--------- | |
| 521 | A D E I G L D N D K A F V V G G T G L A | 540 |
| 1621 | GATGCTATGAGTATAGCTCCAGTTGCTTCTCAACTTAAAGATGGAGATGCTACTCCAATA | 1680 |
| | ---------+---------+---------+---------+---------+--------- | |
| 541 | D A M S I A P V A S Q L K D G D A T P I | 560 |
| 1681 | GTAGTTGTAGATGGAAAAGCAAAAGAAATAAGTGATGATGCTAAGAGTTTCTTAGGAACT | 1740 |
| | ---------+---------+---------+---------+---------+--------- | |
| 561 | V V V D G K A K E I S D D A K S F L G T | 580 |
| 1741 | TCTGATGTTGATATAATAGGTGGAAAAAATAGCGTATCTAAAGAGATTGAAGAGTCAATA | 1800 |
| | ---------+---------+---------+---------+---------+--------- | |
| 581 | S D V D I I G G K N S V S K E I E E S I | 600 |
| 1801 | GATAGTGCAACTGGAAAAACTCCAGATAGAATAAGTGGAGATGATAGACAAGCAACTAAT | 1860 |
| | ---------+---------+---------+---------+---------+--------- | |
| 601 | D S A T G K T P D R I S G D D R Q A T N | 620 |
| 1861 | GCTGAAGTTTTAAAAGAAGATGATTATTTCACAGATGGTGAAGTTGTGAATTACTTTGTT | 1920 |
| | ---------+---------+---------+---------+---------+--------- | |
| 621 | A E V L K E D D Y F T D G E V V N Y F V | 640 |
| 1921 | GCAAAAGATGGTTCTACTAAAGAAGATCAATTAGTAGATGCCTTAGCAGCAGCACCAATA | 1980 |
| | ---------+---------+---------+---------+---------+--------- | |
| 641 | A K D G S T K E D Q L V D A L A A A P I | 660 |
| 1981 | GCAGGTAGATTTAAGGAGTCTCCAGCTCCAATCATACTAGCTACTGATACTTTATCTTCT | 2040 |
| | ---------+---------+---------+---------+---------+--------- | |
| 661 | A G R F K E S P A P I I L A T D T L S S | 680 |
| 2041 | GACCAAAATGTAGCTGTAAGTAAAGCAGTTCCTAAAGATGGTGGAACTAACTTAGTTCAA | 2100 |
| | ---------+---------+---------+---------+---------+--------- | |
| 681 | D Q N V A V S K A V P K D G G T N L V Q | |
| 2101 | GTAGGTAAAGGTATAGCTTCTTCAGTTATAAACAAAATGAAAGATTTATTAGATATG | 2157 |
| | ---------+---------+---------+---------+---------+------- | |
| 701 | V G K G I A S S V I N K M K D L L D M | 719 |

APPENDIX 5

SEQ ID No. 7. Nucleotide sequence of slpA from Clostridium
difficile strain 171862, PCR type 17, with translation. The
putative secretory signal cleavage site (□) and site of
cleavage to form the two mature SLPs (♦) are indicated.

| | | |
|---|---|---|
| 1 | ATGAATAAGAAAAACTTAGCAATGGCTATGGCAGCAGTTACTGTTGTGGGTTCTGCAGCG | 60 |
| | ---------+---------+---------+---------+---------+--------- | |
| 1 | M N K K N L A M A M A A V T V V G S A A | 20 |
| 61 | CCAATATTTGCAGATAGTACTACGCCAGGTTATACTGTAGTGAAAAATGATTGGAAAAAA | 120 |
| | ---------+---------+---------+---------+---------+--------- | |
| 21 | P I F A D S T T P G Y T V V K N D W K K | 40 |
| | □ | |
| 121 | GCAGTAAAACAATTACAAGATGGGTTGAAAAATAAAACTATATCAACAATAAAGGTGTCT | 180 |
| | ---------+---------+---------+---------+---------+--------- | |
| 41 | A V K Q L Q D G L K N K T I S T I K V S | 60 |
| 181 | TTTAATGGAAACTCTGTTGGAGAAGTTACACCAGCCAGTTCTGGAGCAAAAAAAGCAGAT | 240 |
| | ---------+---------+---------+---------+---------+--------- | |
| 61 | F N G N S V G E V T P A S S G A K K A D | 80 |
| 241 | AGAGATGCTGCAGCTGAAAAGTTATATAATTTAGTAAATACACAATTAGATAAACTAGGT | 300 |
| | ---------+---------+---------+---------+---------+--------- | |
| 81 | R D A A A E K L Y N L V N T Q L D K L G | 100 |
| 301 | GATGGAGATTACGTTGACTTTGAAGTAACTTATAATTTAGCTACTCAAATAATTACAAAA | 360 |
| | ---------+---------+---------+---------+---------+--------- | |
| 101 | D G D Y V D F E V T Y N L A T Q I I T K | 120 |
| 361 | GCAGAAGCAGAGGCAGTTCTTACAAAATTACAACAATATAATGATAAAGTACTTATAAAT | 420 |
| | ---------+---------+---------+---------+---------+--------- | |
| 121 | A E A E A V L T K L Q Q Y N D K V L I N | 140 |
| 421 | TCTGCAACAGATACAGTAAAAGGTATGGTATCTGATACACAAGTTGATAGCAAAAATGTT | 480 |
| | ---------+---------+---------+---------+---------+--------- | |
| 141 | S A T D T V K G M V S D T Q V D S K N V | 160 |
| 481 | GCAGCTAACCCACTTAAAGTTAGTGATATGTATACAATACCATCTGCTATTACTGGAAGT | 540 |
| | ---------+---------+---------+---------+---------+--------- | |
| 161 | A A N P L K V S D M Y T I P S A I T G S | 180 |
| 541 | GATGATTCTGGGTATAGTATTGCTAAACCAACAGAAAAGACTACAaGTTTATTGTATGGT | 600 |
| | ---------+---------+---------+---------+---------+--------- | |
| 181 | D D S G Y S I A K P T E K T T S L L Y G | 200 |
| 601 | ACGGTTGGTGATGCAACTGCAGGTAAAGCAATAACAGTAGATACAGCTTCAAATGAAGCT | 660 |
| | ---------+---------+---------+---------+---------+--------- | |
| 201 | T V G D A T A G K A I T V D T A S N E A | 220 |
| 661 | TTTGCTGGAAATGGAAAGGTTATTGACTACAATAAATCATTCAAAGCAACTGTACAAGGA | 720 |
| | ---------+---------+---------+---------+---------+--------- | |
| 221 | F A G N G K V I D Y N K S F K A T V Q G | 240 |
| 721 | GATGGAACAGTTAAGACAAGCGGGGTTGTACTTAAAGATGCAAGTGATATGGCTGCAACA | 780 |
| | ---------+---------+---------+---------+---------+--------- | |
| 241 | D G T V K T S G V V L K D A S D M A A T | 260 |
| 781 | GGTACTATAAAAGTTAGAGTTACAAGTGCAAAAGAAGAATCTATTGATGTGGATTCAAGT | 840 |
| | ---------+---------+---------+---------+---------+--------- | |
| 261 | G T I K V R V T S A K E E S I D V D S S | 280 |
| 841 | TCATATATTAGTGCTGAAAATTTAGCTAAAAAATATGTATTTAATCCTAAAGAGGTTTCT | 900 |
| | ---------+---------+---------+---------+---------+--------- | |
| 281 | S Y I S A E N L A K K Y V F N P K E V S | 300 |
| 901 | GAAGCTTATAATGCAATAGTTGCATTACAAAATGATGGAATAGAATCTGATTTAGTACAA | 960 |
| | ---------+---------+---------+---------+---------+--------- | |
| 301 | E A Y N A I V A L Q N D G I E S D L V Q | 320 |
| 961 | TTAGTTAATGGAAAATATCAAGTTATTTTCTATCCAGAAGGAAAAAGATTAGAAACTAAA | 1020 |
| | ---------+---------+---------+---------+---------+--------- | |
| 321 | L V N G K Y Q V I F Y P E G K R L E T K | 340 |
| 1021 | TCTGCAGATATAATAGCTGATGCAGATAGTCCAGCTAAAATAACTATAAAAGCTAATAAA | 1081 |
| | ---------+---------+---------+---------+---------+--------- | |
| 341 | S A D I I A D A D S P A K I T I K A N K | 360 |
| | ♦ | |
| 1081 | TTAAAAGATTTAAAAGATTATGTAGATGATTTAAAAACATACAATAATACTTACTCAAAT | 1140 |
| | ---------+---------+---------+---------+---------+--------- | |
| 361 | L K D L K D Y V D D L K T Y N N T Y S N | 380 |
| 1141 | GTTGTAACAGTAGCAGGAGAAGATAGAATAGAAACTGCTATAGAATTAAGTAGTAAATAT | 1200 |
| | ---------+---------+---------+---------+---------+--------- | |
| 381 | V V T V A G E D R I E T A I E L S S K Y | 400 |
| 1201 | TATAATTCTGATGATAAAAATGCAATAACTGATGATGCAGTTAATAATATAGTATTAGTT | 1260 |
| | ---------+---------+---------+---------+---------+--------- | |
| 401 | Y N S D D K N A I T D D A V N N I V L V | 420 |
| 1261 | GGATCTACATCTATAGTTGATGGTCTTGTTGCATCACCATTAGCTTCAGAAAAAACAGCT | 1320 |
| | ---------+---------+---------+---------+---------+--------- | |
| 421 | G S T S I V D G L V A S P L A S E K T A | 440 |
| 1321 | CCATTATTATTAACTTCAAAAGATAAATTAGATTCATCAGTAAAATCTGAGATAAAAAGA | 1380 |
| | ---------+---------+---------+---------+---------+--------- | |
| 441 | P L L L T S K D K L D S S V K S E I K R | 460 |
| 1381 | GTTATGAACTTAAAGAGTGATACTGGTATAAATACTTCTAAAAAAGTTTATTTAGCTGGT | 1440 |
| | ---------+---------+---------+---------+---------+--------- | |
| 461 | V M N L K S D T G I N T S K K V Y L A G | 480 |
| 1441 | GGAGTTAATTCTATATCTAAAGATGTAGAAGATGAATTGAAAAATATGGGCCTTAAAGTT | 1500 |
| | ---------+---------+---------+---------+---------+--------- | |
| 481 | G V N S I S K D V E D E L K N M G L K V | 500 |
| 1501 | ACTAGATTATCAGGAGAAGACAGATACGAAACTTCTTTAGCAATAGCTGATGAAATAGGT | 1560 |
| | ---------+---------+---------+---------+---------+--------- | |
| 501 | T R L S G E D R Y E T S L A I A D E I G | 520 |
| 1561 | CTTGATAATGATAAAGCATTTGTAGTTGGTGGTACTGGATTGGCAGATGCTATGAGTATA | 1620 |
| | ---------+---------+---------+---------+---------+--------- | |
| 521 | L D N D K A F V V G G T G L A D A M S I | 540 |
| 1621 | GCTCCAGTTGCTTCTCAACTTAAAGATGGAGATGCTACTCCAATAGTAGTTGTAGATGGA | 1680 |
| | ---------+---------+---------+---------+---------+--------- | |
| 541 | A P V A S Q L K D G D A T P I V V V D G | 560 |
| 1681 | AAAGCAAAAGAAATAAGTGATGATGCTAAGAGTTTCTTAGGAACTTCTGATGTTGATATA | 1740 |
| | ---------+---------+---------+---------+---------+--------- | |
| 561 | K A K E I S D D A K S F L G T S D V D I | 580 |
| 1741 | ATAGGTGGAAAAAATAGCGTATCTAAAGAGATTGAAGAGTCAATAGATAGTGCAACTGGA | 1800 |
| | ---------+---------+---------+---------+---------+--------- | |
| 581 | I G G K N S V S K E I E E S I D S A T G | 600 |
| 1801 | AAAACTCCAGATAGAATAAGTGGAGATGACAGACAAGCAACTAATGCTGAAGTTTTAAAA | 1860 |
| | ---------+---------+---------+---------+---------+--------- | |
| 601 | K T P D R I S G D D R Q A T N A E V L K | 620 |
| 1861 | GAAGATGATTATTTCAAAGATGGTGAAGTTGTGAATTACTTTGTTGCAAAAGATGGTTCT | 1920 |
| | ---------+---------+---------+---------+---------+--------- | |
| 621 | E D D Y F K D G E V V N Y F V A K D G S | 640 |
| 1921 | ACTAAAGAAGATCAATTAGTAGATGCATTAGCAGCAGCACCAATAGCAGGTAGATTTAAG | 1980 |
| | ---------+---------+---------+---------+---------+--------- | |
| 641 | T K E D Q L V D A L A A A P I A G R F K | 660 |
| 1981 | GAGTCTCCAGCTCCAATCATACTAGCTACTGATACTTTATCTTCTGACCAAAATGTAGCT | 2040 |
| | ---------+---------+---------+---------+---------+--------- | |
| 661 | E S P A P I I L A T D T L S S D Q N V A | 680 |
| 2041 | GTAAGTAAAGCAGTTCCTAAAGATGGTGGAACTAACTTAGTTCAAGTAGGTAAAGGTATA | 2100 |
| | ---------+---------+---------+---------+---------+--------- | |
| 681 | V S K A V P K D G G T N L V Q V G K G I | 700 |
| 2101 | GCTTCTTCAGTTATAAACAAAATGAAAGATTTATTAGATATGTAA | 2145 |
| | ---------+---------+---------+---------+---------+----- | |
| 701 | A S S V I N K M K D L L D M * | 715 |

APPENDIX 6

SEQ ID No 8. Nucleotide sequence of slpA from Clostridium
difficile strain 173644, PCR type 31, with translation. The
putative secretory signal cleavage site (□) and site of
cleavage to form the two mature SLPs (♦) are indicated.

| | | |
|---|---|---|
| 1 | ATGAATAAGAAGGATATAGCAATAGCTATGTCAGGATTAACAGTATTAGCTTCTGCAGCA | 60 |
| | ---------+---------+---------+---------+---------+--------- | |
| 1 | M N K K D I A I A M S G L T V L A S A A | 20 |
| 61 | CCTGTATTTGCTGCTAGTAGTTTTACAGCAGATTATAATTATACTGTAGTGCAAGGAAAA | 120 |
| | ---------+---------+---------+---------+---------+--------- | |
| 21 | P V F A A S S F T A D Y N Y T V V Q G K | 40 |
| | □ | |
| 121 | TATCAAAAAGTTATAACTGGATTACAAGATGGTTTAAAAAATGGAAAAATAACAAATATT | 180 |
| | ---------+---------+---------+---------+---------+--------- | |
| 41 | Y Q K V I T G L Q D G L K N G K I T N I | 60 |
| 181 | GATGTAATATTTGATGGAAGTTCAATTGGTGAGGTAGTGCCAGGTTCTGATGCTGCAGCT | 240 |
| | ---------+---------+---------+---------+---------+--------- | |
| 61 | D V I F D G S S I G E V V P G S D A A A | 80 |
| 241 | GCAGCTACTAAATTAAAAAGTTTAGTTGATGATAAGTTAGATAACTTAGGTGATGGAAAA | 300 |
| | ---------+---------+---------+---------+---------+--------- | |
| 81 | A A T K L K S L V D D K L D N L G D G K | 100 |
| 301 | TACGTTCAATTTAATGTTACTTATACTACTAAATCTATAATAACTAAAGCAGAATTAAAA | 360 |
| | ---------+---------+---------+---------+---------+--------- | |
| 101 | Y V Q F N V T Y T T K S I I T K A E L K | 120 |
| 361 | AATTATTATAATCAATTAGAAAGTAGTAAAGATAGAATACTTATAGGAAATGAACCTCAA | 420 |
| | ---------+---------+---------+---------+---------+--------- | |
| 121 | N Y Y N Q L E S S K D R I L I G N E P Q | 140 |
| 421 | GATACAGGAACTAAAGGTCTTATAAAAGCTGATACTGATGGTACTACTGCTGTTGCAGCA | 480 |
| | ---------+---------+---------+---------+---------+--------- | |
| 141 | D T G T K G L I K A D T D G T T A V A A | 160 |
| 481 | GCTGCACCATTGAAATTATCAGATATATTTACGTTTAGTTATGATGAAGTAACAGGTGTA | 540 |
| | ---------+---------+---------+---------+---------+--------- | |
| 161 | A A P L K L S D I F T F S Y D E V T G V | 180 |
| 541 | CTTAAAGCAGAACCAACAAGTAAAGTAAGCGCTGGTAAAGTTCAAGGTCTAAAATATGGA | 600 |
| | ---------+---------+---------+---------+---------+--------- | |
| 181 | L K A E P T S K V S A G K V Q G L K Y G | 200 |
| 601 | AATACAGGAGCAACTAACTATACTTCTGGAGCTGAAATATCTGTTCCTACTACAGGCTTA | 660 |
| | ---------+---------+---------+---------+---------+--------- | |
| 201 | N T G A T N Y T S G A E I S V P T T G L | 220 |
| 661 | ACATTAACTGCTGATACAACTGCAACAACAGATGTAAATATTTCTGATGTTATGAGTGCA | 720 |
| | ---------+---------+---------+---------+---------+--------- | |
| 221 | T L T A D T T A T T D V N I S D V M S A | 240 |
| 721 | TTTAAATTTAATGGTACTGATACGATTAGTGGATTCCCAGCTGGTTCATCAGCTTCTACT | 780 |
| | ---------+---------+---------+---------+---------+--------- | |
| 241 | F K F N G T D T I S G F P A G S S A S T | 260 |
| 781 | CTTAGAGCAAGTATAAAAGTAATAAATGCAAAAGAAGAATCTATAGATGTTGATTCAAGT | 840 |
| | ---------+---------+---------+---------+---------+--------- | |
| 261 | L R A S I K V I N A K E E S I D V D S S | 280 |
| 841 | TCACATAGAACAGCTGAAGATTTAGCTGAAAAATATGTATTTAAACCAGAAGATGTGAAT | 900 |
| | ---------+---------+---------+---------+---------+--------- | |
| 281 | S H R T A E D L A E K Y V F K P E D V N | 300 |
| 901 | AAAACTTATGAGGCACTGACTGATTTATATAAAGAAGGTATAACAAGTAATCTTATCACT | 960 |
| | ---------+---------+---------+---------+---------+--------- | |
| 301 | K T Y E A L T D L Y K E G I T S N L I T | 320 |
| 961 | CAAGATGGTGGAAAATATCAAGTTGTTTTATTTGCTCAAGGAAAGAGATTAACTACTAAA | 1020 |
| | ---------+---------+---------+---------+---------+--------- | |
| 321 | Q D G G K Y Q V V L F A Q G K R L T T K | 340 |
| 1021 | GGAGCAACTGGAACTTTAGCAGATGAAAATTCTCCTCTTAAAGTAACAATAAAAGCAGAT | 1080 |
| | ---------+---------+---------+---------+---------+--------- | |
| 341 | G A T G T L A D E N S P L K V T I K A D | 360 |
| | ♦ | |
| 1081 | AAAGTAAAAGACTTAAAAGATTATGTTGAAGATTTAAAAAATGCTAACAATGGATATTCA | 1140 |
| | ---------+---------+---------+---------+---------+--------- | |
| 361 | K V K D L K D Y V E D L K N A N N G Y S | 380 |
| 1141 | AATTCTGTTGTTGTAGCAGGTGAAGATAGAATAGAAACAGCAATAGAGTTAAGTAGCAAA | 1200 |
| | ---------+---------+---------+---------+---------+--------- | |
| 381 | N S V V V A G E D R I E T A I E L S S K | 400 |
| 1201 | TACTATAACTCTGATGATGACAATGCAATAACTAAAGATCCAGTTAACAATGTTGTTTTA | 1260 |
| | ---------+---------+---------+---------+---------+--------- | |
| 401 | Y Y N S D D D N A I T K D P V N N V V L | 420 |
| 1261 | GTTGGTTCTCAAGCTGTAGTTGATGGGCTTGTAGCTTCACCTTTAGCATCTGAAAAAAGA | 1320 |
| | ---------+---------+---------+---------+---------+--------- | |
| 421 | V G S Q A V V D G L V A S P L A S E K R | 440 |
| 1321 | GCTCCTTTACTATTAACTTCAGCAGGAAAATTAGATTCAAGTGTTAAAGCTGAGTTGAAA | 1380 |
| | ---------+---------+---------+---------+---------+--------- | |
| 441 | A P L L L T S A G K L D S S V K A E L K | 460 |
| 1381 | AGAGTAATGGATTTAAAATCTACAACAGGTGTAAATACTTCTAAAAAAGTTTACTTAGCT | 1440 |
| | ---------+---------+---------+---------+---------+--------- | |
| 461 | R V M D L K S T T G V N T S K K V Y L A | 480 |
| 1441 | GGTGGAGTAAACTCTATATCTAAAGATGTAGAAAATGAATTAAAAGATATGGGACTTAAA | 1500 |
| | ---------+---------+---------+---------+---------+--------- | |
| 481 | G G V N S I S K D V E N E L K D M G L K | 500 |
| 1501 | GTTACAAGATTATCAGGAGATGATAGATATGAAACTTCTTTAGCTATAGCTGATGAAATA | 1560 |
| | ---------+---------+---------+---------+---------+--------- | |
| 501 | V T R L S G D D R Y E T S L A I A D E I | 520 |
| 1561 | GGTCTTGATAATGATAAAGCTTTTGTAGTTGGAGGAACAGGATTAGCGGATGCTATGAGT | 1620 |
| | ---------+---------+---------+---------+---------+--------- | |
| 521 | G L D N D K A F V V G G T G L A D A M S | 540 |
| 1621 | ATAGCTCCAGTTGCTTCTCAATTAAGAAACTCAAATGGAGAACTTGACTTAAAAGGTGAT | 1680 |
| | ---------+---------+---------+---------+---------+--------- | |
| 541 | I A P V A S Q L R N S N G E L D L K G D | 560 |
| 1681 | GCAACTCCAATAGTAGTTGTTGATGGAAAAGCTAAAGATATAAATTCTGAAGTAAAAGAT | 1740 |
| | ---------+---------+---------+---------+---------+--------- | |
| 561 | A T P I V V V D G K A K D I N S E V K D | 580 |
| 1741 | TTCTTAGATGATTCACAAGTTGATATAATAGGTGGTGTAAATAGTGTTTCTAAAGAAGTA | 1800 |
| | ---------+---------+---------+---------+---------+--------- | |
| 581 | F L D D S Q V D I I G G V N S V S K E V | 600 |
| 1801 | ATGGAAGCAATAGATGATGCTACTGGAAAATCACCTGAGAGATATAGTGGAGAAGATAGA | 1860 |
| | ---------+---------+---------+---------+---------+--------- | |
| 601 | M E A I D D A T G K S P E R Y S G E D R | 620 |
| 1861 | CAAGCAACAAATGCTAAAGTTATAAAAGAAGATGATTTCTTTAAAAATGGAGAAGTTACA | 1920 |
| | ---------+---------+---------+---------+---------+--------- | |
| 621 | Q A T N A K V I K E D D F F K N G E V T | 640 |
| 1921 | AACTTCTTTGTAGCTAAAGATGGTTCAACTAAAGAAGATCAATTAGTAGATGCTTTAGCA | 1980 |
| | ---------+---------+---------+---------+---------+--------- | |
| 641 | N F F V A K D G S T K E D Q L V D A L A | 660 |
| 1981 | GGTGCTGCAATTGCTGGTAACTTTGGTGTAACAGTAGATAATGAAGGAAAACCTACAGTT | 2040 |
| | ---------+---------+---------+---------+---------+--------- | |
| 661 | G A A I A G N F G V T V D N E G K P T V | 680 |
| 2041 | GCTGATAAAAAAGCTTCTCCAGCACCAATTGTTTTAGCAACAGATTCTTTATCTTCTGAT | 2100 |
| | ---------+---------+---------+---------+---------+--------- | |
| 681 | A D K K A S P A P I V L A T D S L S S D | 700 |
| 2101 | CAAAATGTAGCTATAAGTAAAGCTGTAAATGATGACGCTAATACTAAGAATCTAGTTCAA | 2160 |
| | ---------+---------+---------+---------+---------+--------- | |
| 701 | Q N V A I S K A V N D D A N T K N L V Q | 720 |
| 2161 | GTTGGTAAAGGTATAGCTACTTCAGTTGTAAGTAAAATAAAAGATTTATTAGATATG | 2217 |
| | ---------+---------+---------+---------+---------+------- | |
| 721 | V G K G I A T S V V S K I K D L L D M | 739 |

APPENDIX 7

SEQ ID No 9. Nucleotide sequence of slpA from Clostridium
difficile strain 170444, PCR type 46, with translation. The
putative secretory signal cleavage site (□) and site of
cleavage to form the two mature SLPs (♦) are indicated.

| | | |
|---|---|---|
| 1 | ATGAATAAGAAAAATATAGCAATAGCTATGTCAGGTTTAACAGTTTTAGCTTCGGCTGCT | 60 |
| | ---------+---------+---------+---------+---------+--------- | |
| 1 | M N K K N I A I A M S G L T V L A S A A | 20 |
| 61 | CCTGTTTTTGCTGCAACTACTGGAACACAAGGTTATACTGTAGTTAAAAACGACTGGAAA | 120 |
| | ---------+---------+---------+---------+---------+--------- | |
| 21 | P V F A A T T G T Q G Y T V V K N D W K | 40 |
| | □ | |
| 121 | AAAGCAGTAAAACAATTACAAGATGGACTAAAAGATAATAGTATAGGAAAGATAACTGTA | 180 |
| | ---------+---------+---------+---------+---------+--------- | |
| 41 | K A V K Q L Q D G L K D N S I G K I T V | 60 |
| 181 | TCTTTTAATGATGGGGTTGTGGGTGAAGTAGCTCCTAAAAGTGCTAATAAGAAAGCGGAC | 240 |
| | ---------+---------+---------+---------+---------+--------- | |
| 61 | S F N D G V V G E V A P K S A N K K A D | 80 |
| 241 | AGAGATGCTGCAGCTGAGAAGTTATATAATCTTGTTAACACTCAATTAGATAAATTAGGT | 300 |
| | ---------+---------+---------+---------+---------+--------- | |
| 81 | R D A A A E K L Y N L V N T Q L D K L G | 100 |
| 301 | GATGGAGATTATGTTGATTTTTCTGTAGATTATAATTTAGAAAAAAAAATAATAACTAAT | 360 |
| | ---------+---------+---------+---------+---------+--------- | |
| 101 | D G D Y V D F S V D Y N L E K K I I T N | 120 |
| 361 | CAAGCAGATGCAGAAGCAATTGTTACAAAGTTAAATTCACTTAATGAGAAAACTCTTATT | 420 |
| | ---------+---------+---------+---------+---------+--------- | |
| 121 | Q A D A E A I V T K L N S L N E K T L I | 140 |
| 421 | GATATAGCAACTAAAGATACTTTTGGAATGGTTAGTAAAACACAAGATAGTGAAGGTAAA | 480 |
| | ---------+---------+---------+---------+---------+--------- | |
| 141 | D I A T K D T F G M V S K T Q D S E G K | 160 |
| 481 | AATGTTGCTGCAACAAAGGCACTTAAAGTTAAAGATGTTGCTACATTTGGTTTGAAGTCT | 540 |
| | ---------+---------+---------+---------+---------+--------- | |
| 161 | N V A A T K A L K V K D V A T F G L K S | 180 |
| 541 | GGTGGAAGCGAAGATACTGGATATGTTATTGAAATGAAAGCAGGAGCTGTAGAGGATAAG | 600 |
| | ---------+---------+---------+---------+---------+--------- | |
| 181 | G G S E D T G Y V I E M K A G A V E D K | 200 |
| 601 | TATGGTAAAGTTGGAGATAGTACGGCAGGTATTGCAATAAATCTTCCTAGTACTGGACTT | 660 |
| | ---------+---------+---------+---------+---------+--------- | |
| 201 | Y G K V G D S T A G I A I N L P S T G L | 220 |
| 661 | GAATATGCAGGTAAAGGAACAACAATTGATTTTAATAAAACTTTAAAAGTTGATGTAACA | 720 |
| | ---------+---------+---------+---------+---------+--------- | |
| 221 | E Y A G K G T T I D F N K T L K V D V T | 240 |
| 721 | GGTGGTTCAACACCTAGTGCTGTAGCTGTAAGTGGTTTTGTAACTAAAGATGATACTGAT | 780 |
| | ---------+---------+---------+---------+---------+--------- | |
| 241 | G G S T P S A V A V S G F V T K D D T D | 260 |
| 781 | TTAGCAAAATCAGGTACTATAAATGTAAGAGTTATAAATGCAAAAGAAGAATCAATTGAT | 840 |
| | ---------+---------+---------+---------+---------+--------- | |
| 261 | L A K S G T I N V R V I N A K E E S I D | 280 |
| 841 | ATAGATGCAAGCTCATATACATCAGCTGAAAATTTAGCTAAAAGACATGTATTTGATCCA | 900 |
| | ---------+---------+---------+---------+---------+--------- | |
| 281 | I D A S S Y T S A E N L A K R H V F D P | 300 |
| 901 | GATGAAATTTCTGAAGCATATAAGGCAATAGTAGCATTACAAAATGATGGTATAGAGTCT | 960 |
| | ---------+---------+---------+---------+---------+--------- | |
| 301 | D E I S E A Y K A I V A L Q N D G I E S | 320 |
| 961 | AATTTAGTTCAGTTAGTTAATGGAAAATATCAAGTGATTTTTTATCCAGAAGGTAAAAGA | 1020 |
| | ---------+---------+---------+---------+---------+--------- | |
| 321 | N L V Q L V N G K Y Q V I F Y P E G K R | 340 |
| 1021 | TTAGAAACTAAATCAGCAAATGATACAATAGCTAGTCAAGATACACCAGCTAAAGTAGTT | 1080 |
| | ---------+---------+---------+---------+---------+--------- | |
| 341 | L E T K S A N D T I A S Q D T P A K V V | 360 |
| | ♦ | |
| 1081 | ATAAAAGCTAATAAATTAAAAGATTTAAAAGATTATGTAGATGATTTAAAAACATATAAT | 1140 |
| | ---------+---------+---------+---------+---------+--------- | |
| 361 | I K A N K L K D L K D Y V D D L K T Y N | 380 |
| 1141 | AATACTTATTCAAATGTTGTAACAGTAGCAGGAGAAGATAGAATAGAAACTGCTATAGAA | 1200 |
| | ---------+---------+---------+---------+---------+--------- | |
| 381 | N T Y S N V V T V A G E D R I E T A I E | 400 |
| 1201 | TTAAGTAGTAAATATTATAATTCTGATGATAAAAATGCAATAACTGATAAAGCAGTTAAT | 1260 |
| | ---------+---------+---------+---------+---------+--------- | |
| 401 | L S S K Y Y N S D D K N A I T D K A V N | 420 |
| 1261 | GATATAGTATTAGTTGGATCTACATCTATAGTTGATGGTCTTGTTGCATCACCATTAGCT | 1320 |
| | ---------+---------+---------+---------+---------+--------- | |
| 421 | D I V L V G S T S I V D G L V A S P L A | 440 |
| 1321 | TCAGAAAAAACAGCTCCATTATTATTAACTTCAAAAGATAAATTAGATTCATCAGTAAAA | 1380 |
| | ---------+---------+---------+---------+---------+--------- | |
| 441 | S E K T A P L L L T S K D K L D S S V K | 460 |
| 1381 | TCTGAAATAAAGAGAGTTATGAACTTAAAGAGTGACACTGGTATAAATACTTCTAAAAAA | 1440 |
| | ---------+---------+---------+---------+---------+--------- | |
| 461 | S E I K R V M N L K S D T G I N T S K K | 480 |
| 1441 | GTTTATTTAGCTGGTGGAGTTAATTCTATATCTAAAGATGTAGAAAATGAATTGAAAAAC | 1500 |
| | ---------+---------+---------+---------+---------+--------- | |
| 481 | V Y L A G G V N S I S K D V E N E L K N | 500 |
| 1501 | ATGGGTCTTAAAGTTACTAGATTATCAGGAGAAGACAGATACGAAACTTCTTTAGCAATA | 1560 |
| | ---------+---------+---------+---------+---------+--------- | |
| 501 | M G L K V T R L S G E D R Y E T S L A I | 520 |
| 1561 | GCTGATGAAATAGGTCTTGATAATGATAAAGCATTTGTAGTTGGTGGTACTGGATTAGCA | 1620 |
| | ---------+---------+---------+---------+---------+--------- | |
| 521 | A D E I G L D N D K A F V V G G T G L A | 540 |
| 1621 | GATGCTATGAGTATAGCTCCAGTTGCTTCTCAACTTAAAGATGGAGATGCTACTCCAATA | 1680 |
| | ---------+---------+---------+---------+---------+--------- | |
| 541 | D A M S I A P V A S Q L K D G D A T P I | 560 |
| 1681 | GTAGTTGTAGATGGAAAAGCAAAAGAAATAAGTGATGATGCTAAGAGTTTCTTAGGAACT | 1740 |
| | ---------+---------+---------+---------+---------+--------- | |
| 561 | V V V D G K A K E I S D D A K S F L G T | 580 |
| 1741 | TCTGATGTTGATATAATAGGTGGAAAAAATAGCGTATCTAAAGAGATTGAAGAGTCAATA | 1800 |
| | ---------+---------+---------+---------+---------+--------- | |
| 581 | S D V D I I G G K N S V S K E I E E S I | 600 |
| 1801 | GATAGTGCAACTGGAAAAACTCCAGATAGAATAAGTGGAGATGATAGACAAGCAACTAAT | 1860 |
| | ---------+---------+---------+---------+---------+--------- | |
| 601 | D S A T G K T P D R I S G D D R Q A T N | 620 |
| 1861 | GCTGAAGTTTTAAAAGAAGATGATTATTTCACAGATGGTGAAGTTGTGAATTACTTTGTT | 1920 |
| | ---------+---------+---------+---------+---------+--------- | |
| 621 | A E V L K E D D Y F T D G E V V N Y F V | |
| 1921 | GCAAAAGATGGTTCTACTAAAGAAGATCAATTAGTAGATGCCTTAGCAGCAGCACCAATA | 1980 |
| | ---------+---------+---------+---------+---------+--------- | |
| 641 | A K D G S T K E D Q L V D A L A A A P I | 660 |
| 1981 | GCAGGTAGATTTAAGGAGTCTCCAGCTCCAATCATACTAGCTACTGATACTTTATCTTCT | 2040 |
| | ---------+---------+---------+---------+---------+--------- | |
| 661 | A G R F K E S P A P I I L A T D T L S S | 680 |
| 2041 | GACCAAAATGTAGCTGTAAGTAAAGCAGTTCCTAAAGATGGTGGAACTAACTTAGTTCAA | 2100 |
| | ---------+---------+---------+---------+---------+--------- | |
| 681 | D Q N V A V S K A V P K D G G T N L V Q | 700 |
| 2101 | GTAGGTAAAGGTATAGCTTCTTCAGTTATAAACAAAATGAAAGATTTATTAGATATG | 2157 |
| | ---------+---------+---------+---------+---------+------- | |
| 701 | V G K G I A S S V I N K M K D L L D M | 719 |

APPENDIX 8

SEQ ID No 10. Nucleotide sequence of slpA from Clostridium
difficile strain 170426, PCR type 92, with translation. The
putative secretory signal cleavage site (□) and site of
cleavage to form the two mature SLPs (♦) are indicated.

| | | |
|---|---|---|
| 1 | ATGAATAAGAAAAATATAGCAATAGCTATGTCAGGTTTAACAGTTTTAGCTTCGGCTGCT | 60 |
| | ---------+---------+---------+---------+---------+--------- | |
| 1 | M N K K N I A I A M S G L T V L A S A A | 20 |
| 61 | CCTGTTTTTGCTGCAACTACTGGAACACAAGGTTATACTGTAGTTAAAAACGACTGGAAA | 120 |
| | ---------+---------+---------+---------+---------+--------- | |
| 21 | P V F A A T T G T Q G Y T V V K N D W K | 40 |
| | □ | |
| 121 | AAAGCAGTAAAACAATTACAGGATGGACTAAAAGATAATAGTATAGGAAAGATAACTGTA | 180 |
| | ---------+---------+---------+---------+---------+--------- | |
| 41 | K A V K Q L Q D G L K D N S I G K I T V | 60 |
| 181 | TCTTTTAATGATGGGGTTGTGGGTGAAGTAGCTCCTAAAAGTGCTAATAAGAAAGCGGAC | 240 |
| | ---------+---------+---------+---------+---------+--------- | |
| 61 | S F N D G V V G E V A P K S A N K K A D | 80 |
| 241 | AGAGATGCTGCAGCTGAGAAGTTATATAATCTTGTTAACACTCAATTAGATAAATTAGGT | 300 |
| | ---------+---------+---------+---------+---------+--------- | |
| 81 | R D A A A E K L Y N L V N T Q L D K L G | 301 |
| 301 | GATGGAGATTATGTTGATTTTTCTGTAGATTATAATTTAGAAAAAAAAATAATAACTAAT | 360 |
| | ---------+---------+---------+---------+---------+--------- | |
| 101 | D G D Y V D F S V D Y N L E K K I I T N | 120 |
| 361 | CAAGCAGATGCAGAAGCAATTGTTACAAAGTTAAATTCACTTAATGAGAAAACTCTTATT | 420 |
| | ---------+---------+---------+---------+---------+--------- | |
| 121 | Q A D A E A I V T K L N S L N E K T L I | 140 |
| 421 | GATATAGCAACTAAAGATACTTTTGGAATGGTTAGTAAAACACAAGATAGTGAAGGTAAA | 480 |
| | ---------+---------+---------+---------+---------+--------- | |
| 141 | D I A T K D T F G M V S K T Q D S E G K | 160 |
| 481 | AATGTTGCTGCAACAAAGGCACTTAAAGTTAAAGATGTTGCTACATTTGGTTTGAAGTCT | 540 |
| | ---------+---------+---------+---------+---------+--------- | |
| 161 | N V A A T K A L K V K D V A T F G L K S | 180 |
| 541 | GGTGGAAGCGAAGATACTGGATATGTTGTTGAAATGAAAGCAGGAGCTGTAGAGGATAAG | 600 |
| | ---------+---------+---------+---------+---------+--------- | |
| 181 | G G S E D T G Y V V E M K A G A V E D K | 200 |
| 601 | TATGGTAAAGTTGGAGATAGTACGGCAGGTATTGCAATAAATCTTCCTAGTACTGGACTT | 660 |
| | ---------+---------+---------+---------+---------+--------- | |
| 201 | Y G K V G D S T A G I A I N L P S T G L | 220 |
| 661 | GAATATGCAGGTAAAGGAACAACAATTGATTTTAATAAAACTTTAAAAGTTGATGTAACA | 720 |
| | ---------+---------+---------+---------+---------+--------- | |
| 221 | E Y A G K G T T I D F N K T L K V D V T | 240 |
| 721 | GGTGGTTCAACACCTAGTGCTGTAGCTGTAAGTGGTTTTGTAACTAAAGATGATACTGAT | 780 |
| | ---------+---------+---------+---------+---------+--------- | |
| 241 | G G S T P S A V A V S G F V T K D D T D | 260 |
| 781 | TTAGCAAAATCAGGTACTATAAATGTAAGAGTTATAAATGCAAAAGAAGAATCAATTGAT | 840 |
| | ---------+---------+---------+---------+---------+--------- | |
| 261 | L A K S G T I N V R V I N A K E E S I D | 280 |
| 841 | ATAGATGCAAGCTCATATACATCAGCTGAAAATTTAGCTAAAAGATATGTATTTGATCCA | 900 |
| | ---------+---------+---------+---------+---------+--------- | |
| 281 | I D A S S Y T S A E N L A K R Y V F D P | 300 |
| 901 | GATGAAATTTCTGAAGCATATAAGGCAATAGTAGCATTACAAAATGATGGTATAGAGTCT | 960 |
| | ---------+---------+---------+---------+---------+--------- | |
| 301 | D E I S E A Y K A I V A L Q N D G I E S | 320 |
| 961 | AATTTAGTTCAGTTAGTTAATGGAAAATATCAAGTGATTTTTTATCCAGAAGGTAAAAGA | 1020 |
| | ---------+---------+---------+---------+---------+--------- | |
| 321 | N L V Q L V N G K Y Q V I F Y P E G K R | 340 |
| 1021 | TTAGAAACTAAATCAGCAAATGATACAATAGCTAGTCAAGATACACCAGCTAAAGTAGTT | 1080 |
| | ---------+---------+---------+---------+---------+--------- | |
| 341 | L E T K S A N D T I A S Q D T P A K V V | 360 |
| | ♦ | |
| 1081 | ATAAAAGCTAATAAATTAAAAGATTTAAAAGATTATGTAGATGATTTAAAAACATATAAT | 1140 |
| | ---------+---------+---------+---------+---------+--------- | |
| 361 | I K A N K L K D L K D Y V D D L K T Y N | 380 |
| 1141 | AATACTTATTCAAATGTTGTAACAGTAGCAGGAGAAGATAGAATAGAAACTGCTATAGAA | 1200 |
| | ---------+---------+---------+---------+---------+--------- | |
| 381 | N T Y S N V V T V A G E D R I E T A I E | 400 |
| 1201 | TTAAGTAGTAAATATTATAATTCTGATGATAAAAATGCAATAACTGATAAAGCAGTTAAT | 1260 |
| | ---------+---------+---------+---------+---------+--------- | |
| 401 | L S S K Y Y N S D D K N A I T D K A V N | 420 |
| 1261 | GATATAGTATTAGTTGGATCTACATCTATAGTTGATGGTCTTGTTGCATCACCATTAGCT | 1320 |
| | ---------+---------+---------+---------+---------+--------- | |
| 421 | D I V L V G S T S I V D G L V A S P L A | 440 |
| 1321 | TCAGAAAAAACAGCTCCATTATTATTAACTTCAAAAGATAAATTAGATTCATCAGTAAAA | 1380 |
| | ---------+---------+---------+---------+---------+--------- | |
| 441 | S E K T A P L L L T S K D K L D S S V K | 460 |
| 1381 | TCTGAAATAAAGAGAGTTATGAACTTAAAGAGTGACACTGGTATAAATACTTCTAAAAAA | 1440 |
| | ---------+---------+---------+---------+---------+--------- | |
| 461 | S E I K R V M N L K S D T G I N T S K K | 480 |
| 1441 | GTTTATTTAGCTGGTGGAGTTAATTCTATATCTAAAGATGTAGAAAATGAATTGAAAAAC | 1500 |
| | ---------+---------+---------+---------+---------+--------- | |
| 481 | V Y L A G G V N S I S K D V E N E L K N | 500 |
| 1501 | ATGGGTCTTAAAGTTACTAGATTATCAGGAGAAGACAGATACGAAACTTCTTTAGCAATA | 1560 |
| | ---------+---------+---------+---------+---------+--------- | |
| 501 | M G L K V T R L S G E D R Y E T S L A I | 520 |
| 1561 | GCTGATGAAATAGGTCTTGATAATGATAAAGCATTTGTAGTTGGTGGTACTGGATTAGCA | 1620 |
| | ---------+---------+---------+---------+---------+--------- | |
| 521 | A D E I G L D N D K A F V V G G T G L A | 540 |
| 1621 | GATGCTATGAGTATAGCTCCAGTTGCTTCTCAACTTAAAGATGGAGATGCTACTCCAATA | 1680 |
| | ---------+---------+---------+---------+---------+--------- | |
| 541 | D A M S I A P V A S Q L K D G D A T P I | 560 |
| 1681 | GTAGTTGTAGATGGAAAAGCAAAAGAAATAAGTGATGATGCTAAGAGTTTCTTAGGAACT | 1740 |
| | ---------+---------+---------+---------+---------+--------- | |
| 561 | V V V D G K A K E I S D D A K S F L G T | 580 |
| 1741 | TCTGATGTTGATATAATAGGTGGAAAAAATAGCGTATCTAAAGAGATTGAAGAGTCAATA | 1800 |
| | ---------+---------+---------+---------+---------+--------- | |
| 581 | S D V D I I G G K N S V S K E I E E S I | 600 |
| 1801 | GATAGTGCAACTGGAAAAACTCCAGATAGAATAAGTGGAGATGATAGACAAGCAACTAAT | 1860 |
| | ---------+---------+---------+---------+---------+--------- | |
| 601 | D S A T G K T P D R I S G D D R Q A T N | 620 |
| 1861 | GCTGAAGTTTTAAAAGAAGATGATTATTTCACAGATGGTGAAGTTGTGAATTACTTTGTT | 1920 |
| | ---------+---------+---------+---------+---------+--------- | |
| 621 | A E V L K E D D Y F T D G E V V N Y F V | 640 |
| 1921 | GCAAAAGATGGTTCTACTAAAGAAGATCAATTAGTAGATGCCTTAGCAGCAGCACCAATA | 1980 |
| | ---------+---------+---------+---------+---------+--------- | |
| 641 | A K D G S T K E D Q L V D A L A A A P I | 660 |
| 1981 | GCAGGTAGATTTAAGGAGTCTCCAGCTCCAATCATACTAGCTACTGATACTTTATCTTCT | 2040 |
| | ---------+---------+---------+---------+---------+--------- | |
| 661 | A G R F K E S P A P I I L A T D T L S S | 680 |
| 2041 | GACCAAAATGTAGCTGTAAGTAAAGCAGTTCCTAAAGATGGTGGAACTAACTTAGTTCAA | 2100 |
| | ---------+---------+---------+---------+---------+--------- | |
| 681 | D Q N V A V S K A V P K D G G T N L V Q | 700 |
| 2101 | GTAGGTAAAGGTATAGCTTCTTCAGTTATAAACAAAATGAAAGATTTATTAGATATG | 2157 |
| | ---------+---------+---------+---------+---------+------- | |
| 701 | V G K G I A S S V I N K M K D L L D M | 719 |

1-66. (canceled)

67. A vaccine for the treatment or prophylaxis of C. difficile associated disease, the vaccine comprising a C. difficile gene or a C. difficile peptide/polypeptide or a derivative or fragment or mutant or variant thereof which is immunogenic in humans.

68. A vaccine for the treatment or prophylaxis of C. difficile associated disease, the vaccine comprising a C. difficile gene or C. difficile peptide/polypeptide or a derivative or fragment or mutant or variant thereof to which immunoreactivity is detected in individuals who have recovered from C. difficile infection.

69. A vaccine as claimed in claim 67 wherein the gene encodes a C. difficile surface layer protein, SlpA or variant or homologue thereof.

70. A vaccine as claimed in claim 67 wherein the peptide/polypeptide is a C. difficile surface layer protein, SlpA or variant or homologue thereof.

71. A vaccine as claimed in claim 67 wherein the vaccine comprises a chimeric nucleic acid sequence.

72. A vaccine as claimed in 71 wherein the chimeric nucleic acid sequence is derived from the 5′ end of the gene, encoding the mature N-terminal moiety of SlpA from C. difficile.

73. A vaccine as claimed in claim 67 wherein the vaccine comprises a chimeric peptide/polypeptide.

74. A vaccine as claimed in 73 wherein the amino acid sequence of the chimeric peptide/polypeptide is derived from the mature N-terminal moiety of SlpA from C. difficile.

75. A vaccine as claimed in claim 67 wherein the vaccine contains an amino acid sequence SEQ ID No. 1 or a derivative or fragment or mutant or variant thereof.

76. A vaccine as claimed in claim 67 wherein the vaccine contains an amino acid sequence SEQ ID No. 2 or a derivative or fragment or mutant or variant thereof.

77. A vaccine as claimed in claim 67 wherein the vaccine contains a nucleotide sequence SEQ ID No. 3 or a derivative or fragment or mutant or variant thereof.

78. A vaccine as claimed in claim 67 wherein the vaccine contains a nucleotide sequence SEQ ID No. 4 or a derivative or fragment or mutant or variant thereof.

79. A vaccine as claimed in claim 67 wherein the vaccine contains a nucleotide sequence SEQ ID No. 5 or a derivative or fragment or mutant or variant thereof.

80. A vaccine as claimed in claim 67 wherein the vaccine contains a nucleotide sequence SEQ ID No. 6 or a derivative or fragment or mutant or variant thereof.

81. A vaccine as claimed in claim 67 wherein the vaccine contains a nucleotide sequence SEQ ID No. 7 or a derivative or fragment or mutant or variant thereof.

82. A vaccine as claimed in claim 67 wherein the vaccine contains a nucleotide sequence SEQ ID No. 8 or a derivative or fragment or mutant or variant thereof.

83. A vaccine as claimed in claim 67 wherein the vaccine contains a nucleotide sequence SEQ ID No. 9 or a derivative or fragment or mutant or variant thereof.

84. A vaccine as claimed in claim 67 wherein the vaccine contains a nucleotide sequence SEQ ID No. 10 or a derivative or fragment or mutant or variant thereof.

85. A vaccine as claimed in claim 67 in combination with at least one other C. difficile sub-unit.

86. A vaccine for the treatment or prophylaxis of C. difficile associated disease, the vaccine comprising the mature N-terminal moiety of a surface layer protein, SlpA of C. difficile or variant or homologue thereof which is immunogenic in humans.

87. A vaccine as claimed in claim 86 wherein the N-terminal moiety of SlpA contains an amino acid sequence SEQ ID No. 1.

88. A vaccine as claimed in claim 86 wherein the N-terminal moiety of SlpA contains an amino acid sequence SEQ ID No. 2.

89. A vaccine for the treatment or prophylaxis of C. difficile associated disease, the vaccine comprising an immunodominant epitope derived from a C. difficile gene or a C. difficile peptide/polypeptide or a derivative or fragment or mutant or variant thereof which is immunogenic in humans.

90. A vaccine as claimed in claim 67 comprising a pharmaceutically acceptable carrier.

91. A vaccine as claimed in claim 67 in combination with a pharmacologically suitable adjuvant.

92. A vaccine as claimed in claim 91 wherein the adjuvant is interleukin 12.

93. A vaccine as claimed in claim 91 wherein the adjuvant is a heat shock protein.

94. A vaccine as claimed in claim 67 comprising at least one other pharmaceutical product.

95. A vaccine as claimed in claim 94 wherein the pharmaceutical product is an antibiotic.

96. A vaccine as claimed in claim 95 wherein the antibiotic is selected from one or more metronidazole, amoxycillin, tetracycline or erythromycin, clarithromycin or timidazole.

97. A vaccine as claimed in claim 94 wherein the pharmaceutical product comprises an acid-suppressing agent such as omeprazole or bismuth salts.

98. A vaccine as claimed in claim 67 in a form for oral administration.

99. A vaccine as claimed in claim 67 in a form for intranasal administration.

100. A vaccine as claimed in claim 67 in a form for intravenous administration.

101. A vaccine as claimed in claim 67 in a form for intramuscular administration.

102. A vaccine as claimed in claim 67 including a peptide delivery system.

103. An immunodominant epitope derived from a C. difficile gene or a C. difficile peptide/polypeptide or a derivative or fragment or mutant or variant thereof.

104. An immunodominant epitope as claimed in claim 103 wherein the C. difficile peptide/polypeptide contains an amino acid sequence SEQ ID No. 1 or SEQ ID No. 2 or a derivative or fragment or mutant or variant thereof.

105. An immunodominant epitope as claimed in claim 101 wherein the C. difficile peptide/polypeptide contains an amino acid sequence SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10 or a derivative or fragment or mutant or variant thereof.

106. A chimeric nucleic acid sequence derived from the 5′ end of the slpA gene encoding the mature N-terminal moiety of SlpA from C. difficile which is immunogenic in humans.

107. A chimeric peptide/polypeptide wherein the amino acid sequence of the chimeric peptide/polypeptide is derived from the mature N-terminal moiety of SlpA from C. difficile.

108. A C. difficile peptide comprising SEQ ID No. 1.

109. A C. difficile peptide comprising SEQ ID No. 2.

110. A C. difficile gene comprising SEQ ID No. 3.

111. A C. difficile gene comprising SEQ ID No. 4.

112. A C. difficile gene comprising SEQ ID No. 5.

113. A C. difficile gene comprising SEQ ID No. 6.

114. A C. difficile gene comprising SEQ ID No. 7.

115. A C. difficile gene comprising SEQ ID No. 8.

116. A C. difficile gene comprising SEQ ID No. 9.

117. A C. difficile gene comprising SEQ ID No. 10.

118. The use of a C. difficile gene or a C. difficile peptide/polypeptide or a derivative or fragment or mutant or variant thereof which is immunogenic in humans in the preparation of a medicament for use in a method for the treatment or prophylaxis of C. difficile infection or C. difficile associated disease in a host.

119. The use as claimed in claim 118 wherein the medicament which is prepared is a vaccine.

120. A method for preparing a vaccine for prophylaxis or treatment of C. difficile associated disease, the method comprising;
obtaining a C. difficile gene or a C. difficile peptide/polypeptide or a derivative or fragment or mutant or variant thereof which is immunogenic in humans; and
forming a vaccine preparation comprised of said gene or peptide/polypeptide or derivative or fragment or mutant or variant, which is suitable for administration to a host and which when administered raises an immune response.

121. A method as claimed in claim 120 wherein the C. difficile peptide/polypeptide contains an amino acid sequence SEQ ID No. 1 or SEQ ID No. 2 or a derivative or fragment or mutant or variant thereof.

122. A method as claimed in claim 120 wherein the C. difficile gene contains an amino acid sequence SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10 or a derivative or fragment or mutant or variant thereof.

123. A method for prophylaxis or treatment of C. difficile associated disease, the method comprising;
obtaining a C. difficile gene or a C. difficile peptide/polypeptide or a derivative or fragment or mutant or variant thereof which is immunogenic in humans;
forming a vaccine preparation comprised of said gene or peptide/polypeptide or derivative or fragment or mutant or variant, and
administering the vaccine preparation to a host to raise an immune response.

124. Monoclonal or polyclonal antibodies or fragments thereof, to a C. difficile peptide/polypeptide or a derivative or fragment or mutant or variant thereof which is immunogenic in humans.

125. Monoclonal or polyclonal antibodies or fragments thereof, to C. difficile peptide/polypeptide or a derivative or fragment or mutant or variant thereof to which immunoreactivity is detected in individuals who have recovered from C. difficile infection.

126. Purified antibodies or serum obtained by immunisation of an animal with a vaccine according to claim 67.

127. The use of the antibodies or fragments as claimed in claim 124 in the preparation of a medicament for treatment or prophylaxis of C. difficile infection or C. difficile associated disease.

128. The use of the antibodies or serum as claimed in 126 in the preparation of a medicament for treatment or prophylaxis of C. difficile infection or C. difficile associated disease.

129. The use of the antibodies or fragments or serum as claimed in claim 124 for use in passive immunotherapy for established C. difficile infection.

130. The use of the antibodies or fragment or serum as claimed in claim 124 for the eradication of C. difficile associated disease.

131. Use of interleukin 12 as an adjuvant in C. difficile vaccine.

132. The use of humanised antibodies or serum for passive vaccination of an individual with C. difficile infection.


SEQUENCE LISTING (13 sequences)

SEQ ID NO: 1 - protein, 20 aa, Clostridium difficile
DKTKVETADQGYTVVQSKYK

SEQ ID NO: 2 - protein, 20 aa, Clostridium difficile
ATTGTQGYTVVKNDGKKAVK

SEQ ID NO: 3 - DNA, 2157 bp, Clostridium difficile
atgaataagaaaaatatagcaatagctatgtcaggtttaacagttttagcttcggctgctcctgtttttgctgcaactactggaacacaaggttatactgtagttaaaaacgactggaaaaaagcagtaaaacaattacaggatggactaaaagataatagtataggaaagataactgtatcttttaatgatggggttgtgggtgaagtagctcctaaaagtgctaataagaaagcggacagagatgctgcagctgagaagttatataatcttgttaacactcaattagataaattaggtgatggagattatgttgatttttctgtagattataatttagaaaaaaaaataataactaatcaagcagatgcagaagcaattgttacaaagttaaattcacttaatgagaaaactcttattgatatagcaactaaagatacttttggaatggttagtaaaacacaagatagtgaaggtaaaaatgttgctgcaacaaaggcacttaaagttaaagatgttgctacatttggtttgaagtctggtggaagcgaagatactggatatgttgttgaaatgaaagcaggagctgtagaggataagtatggtaaagttggagatagtacggcaggtattgcaataaatcttcctagtactggacttgaatatgcaggtaaaggaacaacaattgattttaataaaactttaaaagttgatgtaacaggtggttcaacacctagtgctgtagctgtaagtggttttgtaactaaagatgatactgatttagcaaaatcaggtactataaatgtaagagttataaatgcaaaagaagaatcaattgatatagatgcaagctcatatacatcagctgaaaatttagctaaaagatatgtatttgatccagatgaaatttctgaagcatataaggcaatagtagcattacaaaatgatggtatagagtctaatttagttcagttagttaatggaaaatatcaagtgattttttatccagaaggtaaaagattagaaactaaatcagcaaatgatacaatagctagtcaagatacaccagctaaagtagttataaaagctaataaattaaaagatttaaaagattatgtagatgatttaaaaacatataataatacttattcaaatgttgtaacagtagcaggagaagatagaatagaaactgctatagaattaagtagtaaatattataattctgatgataaaaatgcaataactgataaagcagttaatgatatagtattagttggatctacatctatagttgatggtcttgttgcatcaccattagcttcagaaaaaacagctccattattattaacttcaaaagataaattagattcatcagtaaaatctgaaataaagagagttatgaacttaaagagtgacactggtataaatacttctaaaaaagtttatttagctggtggagttaattctatatctaaagatgtagaaaatgaattgaaaaacatgggtcttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgatagacaagcaactaatgctgaagttttaaaagaagatgattatttcacagatggtgaagttgtgaattactttgttgcaaaagatggttctactaaagaagatcaattagtagatgccttagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatg

SEQ ID NO: 4 - DNA, 1830 bp, Clostridium difficle
atgaaaaaaagaaatttagcaatggctatggcagctgttactgtagtaggttctgctgctccagtttttgcagcagcttcagatgtaatatcactacaagatggtacaaatgataagtatacagtatcaaatactaaagctagtgacttagtaaaggatattttagcagcacaaaacttaacaacaggtgcagttattttgaacaaagatacaaaagttactttctatgatgcaaatgagaaagattcttcaactccaactggagataaaaaagtttattcagaacaaactttaactacagctaatggaaatgaagattatgtaaagacaactttaaaaaatttagatgcaggagaatatgctattatagatttaacttataataatgctaaaactgttgaaattaaagtagtagcagctagtgaaaaaacagtagttgtatctagtgatgcgaaaaatagtgcaaaagatatagctgaaaaatatgtgtttgaagacaaagacttagaaaatgcactaaaaactataaatgcctcagatttcagtaaaactgatagttactatcaagtagttctttatccaaaaggaaagagattacaaggtttctcaacttatagagctacaaattataatgaaggaactgcatatggtaatacaccagtaatattaactctaaaatctactagtaagagtaatttaaagactgcagtagaagagttacaaaaattgaatgctagttattctaatactacaactttagctggtgatgacagaatacaaacagctatagagataagtaaagaatattacaataatgatggcgagaaatcagatcattcagctgatgttaaagagaatgttaaaaatgttgtattagtaggtgcaaatgcactagtagatggattagttgcggctcctttagcagcagaaaaagatgctccactattattaacttcaaaagataaattagattcgtcagtaaaatctgaaataaagagagttttagacttaaaaacttcaacagaagtaacaggaaaaacagtttatatagctggtggagttaatagtgtatctaaagaagttgtaacagaattagaatcaatgggattaaaagttgaaagattctcaggtgatgatagatatgaaacttctttaaaaatagcaggtgaaataggcttagataatgataaggcttatgtagttggtggaacaggattagcagatgccatgagtatagcttcagttgcttctactaaattagatggtaatggtgttgtagatagaacaaatggacatgctactccaatagttgttgtagatggaaaagctgataaaatatctgatgacttagatagtttcttaggaagcgctgatgtagatataataggtggatttgcaagtgtatctgaaaagatggaagaagctatatcagatgctactggtaaaggcgttacaagagttaaaggcgacgatagacaagacactaactctgaagttataaaaacatattatgctaatgatactgaaatagctaaagctgcagttttagataaagattcaggtgcttcaagtagtgatgcaggagtatttaatttctatgtagctaaagatggatctacaaaagaagatcaattagttgatgcattagcagtaggagctgttgctggatataaacttgctccagttgtattagctactgattctttatcttctgatcaatcggttgctataagcaaagttgtaggagaaaaatattctaaagatttaacacaagttggtcaaggaatagctaattcagttataaacaaaatgaaagatttattagatatg

SEQ ID NO: 5 - DNA, 2158 bp, Clostridium difficile
atgaataagaaaaatatagcaatagctatgtcaggtttaacagttttagcttcggctgctcctgtttttgctgcaactactggaacacaaggttatactgtagttaaaaacgactggaaaaaagcagtaaaacaattacaagatggactaaaagataatagtataggaaagataactgtatcttttaatgatggggttgtgggtgaagtagctcctaaaagtgctaataagaaagcggacagagatgctgcagctgagaagttatataatcttgttaacactcaattagataaattaggtgatggagattatgttgatttttctgtagattataatttagaaaacaaaataataactaatcaagcagatgcagaagcaattgttacaaagttaaattcacttaatgagaaaactcttattgatatagcaactaaagatacttttggaatggttagtaaaacacaagatagtgaaggtaaaaatgttgctgcaacaaaggcacttaaagttaaagatgttgctacatttggtttgaagtctggtggaagcgaagatactggatatgttgttgaaatgaaagcaggagctgtagaggataagtatggtaaagttggagatagtacggcaggtattgcaataaatcttcctagtactggacttgaatatgcaggtaaaggaacaacaattgattttaataaaactttaaaagttgatgtaacaggtggttcaacacctagtgctgtagctgtaagtggttttgtaactaaagatgatactgatttagcaaaatcaggtactataaatgtaagagttataaatgcaaaagaagaatcaattgatatagatgcaagctcatatacatcagctgaaaatttagctaaaagatatgtatttgatccagatgaaatttctgaagcatataaggcaatagtagcattacaaaatgatggtatagagtctaacttagttcagttagttaatggaaaatatcaagtgattttttatccagaaggtaaaagattagaaactaaatcagcaaatgatacaatagctagtcaagatacaccagctaaagtagttataaaagctaataaattaaaagatttaaaagattatgtagatgatttaaaaacatataataatacttattcaaatgttgtaacagtagcaggagaagatagaatagaaactgctatagaattaagtagtaaatattataattctgatgataaaaatgcaataactgataaagcagttaatgatatagtattagttggatctacatctatagttgatggtcttgttgcatcaccattagcttcagaaaaaacagctccattattattaacttcaaaagataaattagattcatcagtaaaatctgaaataaagagagttatgaacttaaagagtgacactggtataaatacttctaaaaaagtttatttagctggtggagttaattctatatctaaagatgtagaaaatgaattgaaaaacatgggtcttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgatagacaagcaactaatgctgaagttttaaaagaagatgattatttcacagatggtgaagttgtgaattactttgttgcaaaagatggttctactaaagaagatcaattagtagatgccttagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatgg

SEQ ID NO: 6 - DNA, 2271 bp, Clostridium difficile
atgaataagaaaaatatagcaatagctatgtcaggtttaacagttttagcttcggctgcacctgtatttgcagatgatacaaaagttgaaactggtgatcaaggatatacagtggtacaaagcaagtataagaaagctgttgaacaattacaaaaaggaatattagatggaagtataacagaaattaaagttttctttgagggaactttagcatctactataaaagtaggttctgagcttaatgcagcagatgcaagtaaattattgtttacacaagtagataataaactagataatttaggtgatggagattatgtagatttcttaataacttctccaggtcaaggggataaaataactacaagtaaacttgttgcattgaaagatttaacaggtgcttcagcagatgctataattgctggaacatcttcagcagatggtgttgttacaaatactggagctgctagtggttctactgagacaaattcagcaggaacaaaacttgcaatgtcagctatttttgacacagcatatacagattcatctgaaactgcggttaagattactataaaagcagatatgaatgatactaaatttggtaaagcaggtgagacaacttattcaactgggcttacatttgaagatgggtctacagaaaaaattgttaaattaggggacagtgatattatagatataactaaagctcttaaacttactgttgttcctggaagtaaagcaactgttaagtttgctgaaaaaacaccaagtgccagtgttcaaccagtaataacaaagcttagaataataaatgctaaagaagaaacaatagatattgacgctagttctagtaaaacagcacaagatttagctaaaaaatatgtatttaataaaactgatttaaatactctttataaagtattaaatggagatgaagcagatactaatggattaatagaagaagttagtggaaaatatcaagtagttctttatccagaaggaaaaagagttacaactaagagtgctgcaaaggcttcaattgctgatgaaaattcaccagttaaattaactcttaagtcagataagaagaaagacttaaaagattatgtggatgatttaagaacatataataatggatattcaaatgctatagaagtagcaggagaagatagaatagaaactgcaatagcattaagtcaaaaatattataactctgatgatgaaaatgctatatttagagattcagttgataatgtagtattggttggaggaaatgcaatagttgatggacttgtagcttctcctttagcttctgaaaagaaagctcctttattattaacttcaaaagataaattagattcaagcgtaaaagctgaaataaagagagttatgaatataaagagtacaacaggtataaatacttcaaagaaagtttatttagctggtggagttaattctatatctaaagaagtagaaaatgaattaaaagatatgggacttaaagttacaagattagcaggagatgatagatatgaaacttctctaaaaatagctgatgaagtaggtcttgataatgataaagcatttgtagttggaggaacaggattagcagatgccatgagtatagctccagttgcatctcaattaagaaatgctaatggtaaaatggatttagctgatggtgatgctacaccaatagtagttgtagatggaaaagctaaaactataaatgatgatgtaaaagatttcttagatgattcacaagttgatataataggtggagaaaacagtgtatctaaagatgttgaaaatgcaatagatgatgctacaggtaaatctccagatagatatagtggagatgatagacaagcaactaatgcaaaagttataaaagaatcttcttattatcaagataacttaaataatgataaaaaagtagttaatttctttgtagctaaagatggttctactaaagaagatcaattagttgatgctttagcagcagctccagttgcagcaaactttggtgtaactcttaattctgatggtaagccagtagataaagatggtaaagtattaactggttctgataatgataaaaataaattagtatctccagcacctatagtattagctactgattctttatcttcagatcaaagtgtatctataagtaaagttcttgataaagataatggagaaaacttagttcaagttggtaaaggtatagctacttcagttataaacaaaatgaaagatttattagatatgtaa

SEQ ID NO: 7 - DNA, 2158 bp, Clostridium difficile
atgaataagaaaaatatagcaatagctatgtcaggtttaacagttttagcttcggctgctcctgtttttgctgcaactactggaacacaaggttatactgtagttaaaaacgactggaaaaaagcagtaaaacaattacaagatggactaaaagataatagtataggaaagataactgtatcttttaatgatggggttgtgggtgaagtagctcctaaaagtgctaataagaaagcggacagagatgctgcagctgagaagttatataatcttgttaacactcaattagataaattaggtgatggagattatgttgatttttctgtagattataatttagaaaacaaaataataactaatcaagcagatgcagaagcaattgttacaaagttaaattcacttaatgagaaaactcttattgatatagcaactaaagatacttttggaatggttagtaaaacacaagatagtggaggtaaaaatgttgctgcaacaaaggcacttaaagttaaagatgttgctacatttggtttgaagtctggtggaagcgaagatactggatatgttgttgaaatgaaagcaggagctgtagaggataagtatggtaaagttggagatagtacggcaggtattgcaataaatcttcctagtactggacttgaatatgcaggtaaaggaacaacaattgattttaataaaactttaaaagttgatgtaacaggtggttcaacacctagtgctgtagctgtaagtggttttgtaactaaagatgatactgatttagcaaaatcaggtactataaatgtaagagttataaatgcaaaagaagaatcaattgatatagatgcaagctcatatacatcagctgaaaatttagctaaaagatatgtatttgatccagatgaaatttctgaagcatataaggcaatagtagcattacaaaatgatggtatagagtctaatttagttcagttagttaatggaaaatatcaagtgattttttatccagaaggtaaaagattagaaactaaatcagcaaatgatacaatagctagtcaagatacaccagctaaagtagttataaaagctaataaattaaaagatttaaaagattatgtagatgatttaaaaacatataataatacttattcaaatgttgtaacagtagcaggagaagatagaatagaaactgctatagaattaagtagtaaatattataattctgatgataaaaatgcaataactgataaagcagttaatgatatagtattagttggatctacatctatagttgatggtcttgttgcatcaccattagcttcagaaaaaacagctccattattattagcttcaaaagataaattagattcatcagtaaaatctgaaataaagagagttatgaacttaaagagtgacactggtataaatacttctaaaaaagtttatttagctggtggagttaattctatatctaaagatgtagaaaatgaattgaaaaacatgggtcttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgatagacaagcaactaatgctgaagttttaaaagaagatgattatttcacagatggtgaagttgtgaattactttgttgcaaaagatggttctactaaagaagatcaattagtagatgccttagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatgg

SEQ ID NO: 8 - DNA, 2217 bp, Clostridium difficile
atgaataagaaggatatagcaatagctatgtcaggattaacagtattagcttctgcagcacctgtatttgctgctagtagttttacagcagattataattatactgtagtgcaaggaaaatatcaaaaagttataactggattacaagatggtttaaaaaatggaaaaataacaaatattgatgtaatatttgatggaagttcaattggtgaggtagtgccaggttctgatgctgcagctgcagctactaaattaaaaagtttagttgatgataagttagataacttaggtgatggaaaatacgttcaatttaatgttacttatactactaaatctataataactaaagcagaattaaaaaattattataatcaattagaaagtagtaaagatagaatacttataggaaatgaacctcaagatacaggaactaaaggtcttataaaagctgatactgatggtactactgctgttgcagcagctgcaccattgaaattatcagatatatttacgtttagttatgatgaagtaacaggtgtacttaaagcagaaccaacaagtaaagtaagcgctggtaaagttcaaggtctaaaatatggaaatacaggagcaactaactatacttctggagctgaaatatctgttcctactacaggcttaacattaactgctgatacaactgcaacaacagatgtaaatatttctgatgttatgagtgcatttaaatttaatggtactgatacgattagtggattcccagctggttcatcagcttctactcttagagcaagtataaaagtaataaatgcaaaagaagaatctatagatgttgattcaagttcacatagaacagctgaagatttagctgaaaaatatgtatttaaaccagaagatgtgaataaaacttatgaggcactgactgatttatataaagaaggtataacaagtaatcttatcactcaagatggtggaaaatatcaagttgttttatttgctcaaggaaagagattaactactaaaggagcaactggaactttagcagatgaaaattctcctcttaaagtaacaataaaagcagataaagtaaaagacttaaaagattatgttgaagatttaaaaaatgctaacaatggatattcaaattctgttgttgtagcaggtgaagatagaatagaaacagcaatagagttaagtagcaaatactataactctgatgatgacaatgcaataactaaagatccagttaacaatgttgttttagttggttctcaagctgtagttgatgggcttgtagcttcacctttagcatctgaaaaaagagctcctttactattaacttcagcaggaaaattagattcaagtgttaaagctgagttgaaaagagtaatggatttaaaatctacaacaggtgtaaatacttctaaaaaagtttacttagctggtggagtaaactctatatctaaagatgtagaaaatgaattaaaagatatgggacttaaagttacaagattatcaggagatgatagatatgaaacttctttagctatagctgatgaaataggtcttgataatgataaagcttttgtagttggaggaacaggattagcggatgctatgagtatagctccagttgcttctcaattaagaaactcaaatggagaacttgacttaaaaggtgatgcaactccaatagtagttgttgatggaaaagctaaagatataaattctgaagtaaaagatttcttagatgattcacaagttgatataataggtggtgtaaatagtgtttctaaagaagtaatggaagcaatagatgatgctactggaaaatcacctgagagatatagtggagaagatagacaagcaacaaatgctaaagttataaaagaagatgatttctttaaaaatggagaagttacaaacttctttgtagctaaagatggttcaactaaagaagatcaattagtagatgctttagcaggtgctgcaattgctggtaactttggtgtaacagtagataatgaaggaaaacctacagttgctgataaaaaagcttctccagcaccaattgttttagcaacagattctttatcttctgatcaaaatgtagctataagtaaagctgtaaatgatgacgctaatactaagaatctagttcaagttggtaaaggtatagctacttcagttgtaagtaaaataaaagatttattagatatg

SEQ ID NO: 9 - DNA, 2145 bp, Clostridium difficile
atgaataagaaaaacttagcaatggctatggcagcagttactgttgtgggttctgcagcgccaatatttgcagatagtactacgccaggttatactgtagtgaaaaatgattggaaaaaagcagtaaaacaattacaagatgggttgaaaaataaaactatatcaacaataaaggtgtcttttaatggaaactctgttggagaagttacaccagccagttctggagcaaaaaaagcagatagagatgctgcagctgaaaagttatataatttagtaaatacacaattagataaactaggtgatggagattacgttgactttgaagtaacttataatttagctactcaaataattacaaaagcagaagcagaggcagttcttacaaaattacaacaatataatgataaagtacttataaattctgcaacagatacagtaaaaggtatggtatctgatacacaagttgatagcaaaaatgttgcagctaacccacttaaagttagtgatatgtatacaataccatctgctattactggaagtgatgattctgggtatagtattgctaaaccaacagaaaagactacaagtttattgtatggtacggttggtgatgcaactgcaggtaaagcaataacagtagatacagcttcaaatgaagcttttgctggaaatggaaaggttattgactacaataaatcattcaaagcaactgtacaaggagatggaacagttaagacaagcggggttgtacttaaagatgcaagtgatatggctgcaacaggtactataaaagttagagttacaagtgcaaaagaagaatctattgatgtggattcaagttcatatattagtgctgaaaatttagctaaaaaatatgtatttaatcctaaagaggtttctgaagcttataatgcaatagttgcattacaaaatgatggaatagaatctgatttagtacaattagttaatggaaaatatcaagttattttctatccagaaggaaaaagattagaaactaaatctgcagatataatagctgatgcagatagtccagctaaaataactataaaagctaataaattaaaagatttaaaagattatgtagatgatttaaaaacatacaataatacttactcaaatgttgtaacagtagcaggagaagatagaatagaaactgctatagaattaagtagtaaatattataattctgatgataaaaatgcaataactgatgatgcagttaataatatagtattagttggatctacatctatagttgatggtcttgttgcatcaccattagcttcagaaaaaacagctccattattattaacttcaaaagataaattagattcatcagtaaaatctgagataaaaagagttatgaacttaaagagtgatactggtataaatacttctaaaaaagtttatttagctggtggagttaattctatatctaaagatgtagaagatgaattgaaaaatatgggccttaaagttactagattatcaggagaagacagatacgaaacttctttagcaatagctgatgaaataggtcttgataatgataaagcatttgtagttggtggtactggattggcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgacagacaagcaactaatgctgaagttttaaaagaagatgattatttcaaagatggtgaagttgtgaattactttgttgcaaaagatggttctactaaagaagatcaattagtagatgcattagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatgtaa

SEQ ID NO: 10 - DNA, 2158 bp, Clostridium difficile
atgaataagaaaaatatagcaatagctatgtcaggtttaacagttttagcttcggctgctcctgtttttgctgcaactactggaacacaaggttatactgtagttaaaaacgactggaaaaaagcagtaaaacaattacaagatggactaaaagataatagtataggaaagataactgtatcttttaatgatggggttgtgggtgaagtagctcctaaaagtgctaataagaaagcggacagagatgctgcagctgagaagttatataatcttgttaacactcaattagataaattaggtgatggagattatgttgatttttctgtagattataatttagaaaaaaaaataataactaatcaagcagatgcagaagcaattgttacaaagttaaattcacttaatgagaaaactcttattgatatagcaactaaagatacttttggaatggttagtaaaacacaagatagtgaaggtaaaaatgttgctgcaacaaaggcacttaaagttaaagatgttgctacatttggtttgaagtctggtggaagcgaagatactggatatgttattgaaatgaaagcaggagctgtagaggataagtatggtaaagttggagatagtacggcaggtattgcaataaatcttcctagtactggacttgaatatgcaggtaaaggaacaacaattgattttaataaaactttaaaagttgatgtaacaggtggttcaacacctagtgctgtagctgtaagtggttttgtaactaaagatgatactgatttagcaaaatcaggtactataaatgtaagagttataaatgcaaaagaagaatcaattgatatagatgcaagctcatatacatcagctgaaaatttagctaaaagacatgtatttgatccagatgaaatttctgaagcatataaggcaatagtagcattacaaaatgatggtatagagtctaatttagttcagttagttaatggaaaatatcaagtgattttttatccagaaggtaaaagattagaaactaaatcagcaaatgatacaatagctagtcaagatacaccagctaaagtagttataaaagctaataaattaaaagatttaaaagattatgtagatgatttaaaaacatataataatacttattcaaatgttgtaacagtagcaggagaagatagaatagaaactgctatagaattaagtagtaaatattataattctgatgataaaaatgcaataactgataaagcagttaatgatatagtattagttggatctacatctatagttgatggtcttgttgcatcaccattagcttcagaaaaaacagctccattattattaacttcaaaagataaattagattcatcagtaaaatctgaaataaagagagttatgaacttaaagagtgacactggtataaatacttctaaaaaagtttatttagctggtggagttaattctatatctaaagatgtagaaaatgaattgaaaaacatgggtcttaaagttactagattatcaggagaagacagatacgaaacttctttagcaatagctgatgaaataggtcttgataatgataaagcatttgtagttggtggtactggattagcagatgctatgagtatagctccagttgcttctcaacttaaagatggagatgctactccaatagtagttgtagatggaaaagcaaaagaaataagtgatgatgctaagagtttcttaggaacttctgatgttgatataataggtggaaaaaatagcgtatctaaagagattgaagagtcaatagatagtgcaactggaaaaactccagatagaataagtggagatgatagacaagcaactaatgctgaagttttaaaagaagatgattatttcacagatggtgaagttgtgaattactttgttgcaaaagatggttctactaaagaagatcaattagtagatgccttagcagcagcaccaatagcaggtagatttaaggagtctccagctccaatcatactagctactgatactttatcttctgaccaaaatgtagctgtaagtaaagcagttcctaaagatggtggaactaacttagttcaagtaggtaaaggtatagcttcttcagttataaacaaaatgaaagatttattagatatga

SEQ ID NO: 11 - DNA, 23 bp, artificial sequence
  forward primer derived from C. difficile
atggattattatagagatgtgag

SEQ ID NO: 12 - DNA, 31 bp, artificial sequence
  primer based on the downstream sequence derived from Clostridium difficile
ctatttaaagttttattaaaacttatattac

SEQ ID NO: 13 - DNA, 38 bp, artificial sequence
  primer based on end of ORF and the nonsense codon derived from Clostridium difficile
ttacatatctaataaatctttcattttgtttataactg